(19) United States
(12) Patent Application Publication
(10) Pub. No.: US 2024/0013863 A1
(43) Pub. Date: Jan. 11, 2024

(54) INFORMATION PROCESSING APPARATUS, INFORMATION PROCESSING METHOD, AND PROGRAM

(71) Applicant: SONY GROUP CORPORATION, TOKYO (JP)

(72) Inventors: SATOSHI KAWATA, TOKYO (JP); YUTA SAKURAI, TOKYO (JP)

(21) Appl. No.: 18/254,883

(22) PCT Filed: Nov. 8, 2021
(86) PCT No.: PCT/JP2021/040948

§ 371 Date: May 30, 2023

(30) Foreign Application Priority Data
Dec. 4, 2020 (JP) 2020-202081

(51) Int. Cl. G16B 40/00; G16B 15/00; G06N 3/084

(52) U.S. Cl. CPC G16B 40/00; G16B 15/00; G06N 3/084

(57) ABSTRACT

An information processing apparatus according to an embodiment of the present technology includes: an acquisition unit; an inversion unit; and a generation unit. The acquisition unit acquires sequence information relating to a genome sequence. The inversion unit generates, on the basis of the sequence information, inversion information in which the sequence is inverted. The generation unit generates, on the basis of the inversion information, protein information relating to a protein. In this information processing apparatus, sequence information relating to a genome sequence is acquired by the acquisition unit. Further, inversion information in which the sequence is inverted is generated by the inversion unit on the basis of the sequence information. Further, protein information relating to a protein is generated by the generation unit on the basis of the inversion information. As a result, it is possible to predict information relating to a protein with high accuracy.

### TECHNICAL FIELD

The present technology relates to an information processing apparatus, an information processing method, and a program that are applicable to prediction of a three-dimensional structure of a protein, and the like.

### BACKGROUND ART

Patent Literature 1 discloses a machine learning algorithm for predicting a distance map indicating the distance between amino acid residues forming a protein. In this machine learning algorithm, a distance map is predicted by a neural network using the sequence of amino acids contained in the protein and the feature amount of the amino acid sequence as inputs, and output.

### CITATION LIST

### Patent Literature

Patent Literature 1: WO 2020/058176

### DISCLOSURE OF INVENTION

### Technical Problem

There is a demand for a technology capable of predicting the three-dimensional structure of a protein, and the like, with high accuracy.

In view of the circumstances as described above, it is an object of the present technology to provide an information processing apparatus, an information processing method, and a program that are capable of predicting information relating to a protein with high accuracy.

In order to achieve the above-mentioned object, an information processing apparatus according to an embodiment of the present technology includes: an acquisition unit; an inversion unit; and a generation unit.

The acquisition unit acquires sequence information relating to a genome sequence.

The inversion unit generates, on the basis of the sequence information, inversion information in which the sequence is inverted.

The generation unit generates, on the basis of the inversion information, protein information relating to a protein.

In this information processing apparatus, sequence information relating to a genome sequence is acquired by the acquisition unit. Further, inversion information in which the sequence is inverted is generated by the inversion unit on the basis of the sequence information. Further, protein information relating to a protein is generated by the generation unit on the basis of the inversion information. As a result, it is possible to predict information relating to a protein with high accuracy.

The sequence information may be information relating to at least one of a sequence of amino acids, a sequence of DNA, or a sequence of RNA.

The generation unit may include a first prediction unit that predicts first protein information on the basis of the sequence information, a second prediction unit that predicts second protein information on the basis of the inversion information, and an integration unit that integrates the first protein information and the second protein information to generate the protein information.

The protein information may include at least one of a structure of the protein or a function of the protein.

The protein information may include at least one of a contact map indicating a bond between amino acid residues forming the protein, a distance map indicating a distance between amino acid residues forming the protein, or a tertiary structure of the protein.

The integration unit may execute machine learning using the first protein information and the second protein information as inputs to predict the protein information.

The first prediction unit may execute machine learning using the sequence information as an input to predict the first protein information, and the second prediction unit may execute machine learning using the inversion information as an input to predict the second protein information.

The integration unit may include a machine learning model for integration trained on the basis of an error between the protein information predicted using the first protein information for learning predicted using the sequence information for learning associated with correct answer data as a input and the second protein information for learning predicted using the inversion information generated on the basis of the sequence information for learning as an input as inputs and the correct answer data.

The first prediction unit may include a first machine learning model trained on the basis of an error between the first protein information for learning and the correct answer data. In this case, the first machine learning model may be re-trained on the basis of an error between the protein information predicted using the first protein information for learning and the second protein information for learning as inputs and the correct answer data.

The second prediction unit may include a second machine learning model trained on the basis of an error between the second protein information for learning and the correct answer data. In this case, the second machine learning model may be re-trained on the basis of an error between the protein information predicted using the first protein information for learning and the second protein information for learning as inputs and the correct answer data.

The information processing apparatus may further include a feature amount calculation unit that calculates a feature amount on the basis of the sequence information. In this case, the generation unit may generate the protein information on the basis of the feature amount.

The feature amount calculation unit may calculate a first feature amount on the basis of the sequence information, the first prediction unit may predict the first protein information on the basis of the sequence information and the first feature amount, and the second prediction unit may predict the second protein information on the basis of the inversion information and the first feature amount.

The feature amount calculation unit may calculate a first feature amount on the basis of the sequence information and calculate a second feature amount on the basis of the inversion information, the first prediction unit may predict the first protein information on the basis of the sequence information and the first feature amount, and the second prediction unit may predict the second protein information on the basis of the inversion information and the second feature amount.

The first prediction unit may include a first machine learning model trained on the basis of an error between the first protein information predicted using the sequence information for learning, which is associated with correct answer data, and the first feature amount for learning, which is calculated on the basis of the sequence information for learning, as inputs and the correct answer data.

The second prediction unit may include a second machine learning model trained on the basis of an error between the second protein information predicted using the inversion information generated on the basis of the sequence information for learning and the first feature amount for learning, which is calculated on the basis of the sequence information for learning, as inputs and the correct answer data.

The second prediction unit may include a second machine learning model trained on the basis of an error between the second protein information predicted using the inversion information, which is generated on the basis of the sequence information for learning, and the second feature amount for learning calculated on the basis of the inversion information as inputs and the correct answer data.

The feature amount may include at least one of a secondary structure of the protein, annotation information relating to the protein, the degree of catalyst contact of the protein, or a mutual potential between amino acid residues forming the protein.

The sequence information may be information indicating a bonding order from an N-terminal side of amino acid residues forming the protein, and the inversion information may be information indicating a bonding order from a C-terminal side of amino acid residues forming the protein.

An information processing method according to an embodiment of the present technology is an information processing method executed by a computer system, including: acquiring sequence information relating to a genome sequence.

On the basis of the sequence information, inversion information in which the sequence is inverted is generated.

On the basis of the inversion information, first protein information relating to a protein is predicted.

A program according to an embodiment of the present invention causes a computer system to execute the following Steps of:
acquiring sequence information relating to a genome sequence;
generating, on the basis of the sequence information, inversion information in which the sequence is inverted; and
predicting, on the basis of the inversion information, first protein information relating to a protein.

### BRIEF DESCRIPTION OF DRAWINGS

FIG. 1 is a schematic diagram showing a configuration example of a protein analysis system according to an embodiment of the present technology.

FIG. 2 is a flowchart showing an example of generating protein information by the protein analysis system.

FIG. 3 is a schematic diagram showing an example of sequence information.

FIG. 4 is a schematic diagram for describing inversion information.

FIG. 5 is a schematic diagram for describing protein information.

FIG. 6 is a block diagram showing a functional configuration example of an information processing apparatus according to a first embodiment.

FIG. 7 is a schematic diagram showing an example of a machine learning model in a first prediction unit.

FIG. 8 is a schematic diagram for describing training of the machine learning model using teaching data in the first prediction unit.

FIG. 9 is a schematic diagram showing an example of a machine learning model in a second prediction unit.

FIG. 10 is a schematic diagram showing an example of a machine learning model in an integration unit.

FIG. 11 is a schematic diagram for describing training of the machine learning model in the integration unit.

FIG. 12 is a schematic diagram for describing an error of protein information.

FIG. 13 is a block diagram showing a functional configuration example of an information processing apparatus according to a second embodiment.

FIG. 14 is a schematic diagram for describing calculation of a feature amount.

FIG. 15 is a schematic diagram showing an example of a machine learning model in a first prediction unit.

FIG. 16 is a schematic diagram for describing training of the machine learning model using teaching data in the first prediction unit.

FIG. 17 is a block diagram showing a functional configuration example of an information processing apparatus according to a third embodiment.

FIG. 18 is a block diagram showing an example of a hardware configuration of a computer capable of realizing an information processing apparatus.

### MODE(S) FOR CARRYING OUT THE INVENTION

Hereinafter, embodiments according to the present technology will be described with reference to the drawings.

### [Protein Analysis System]

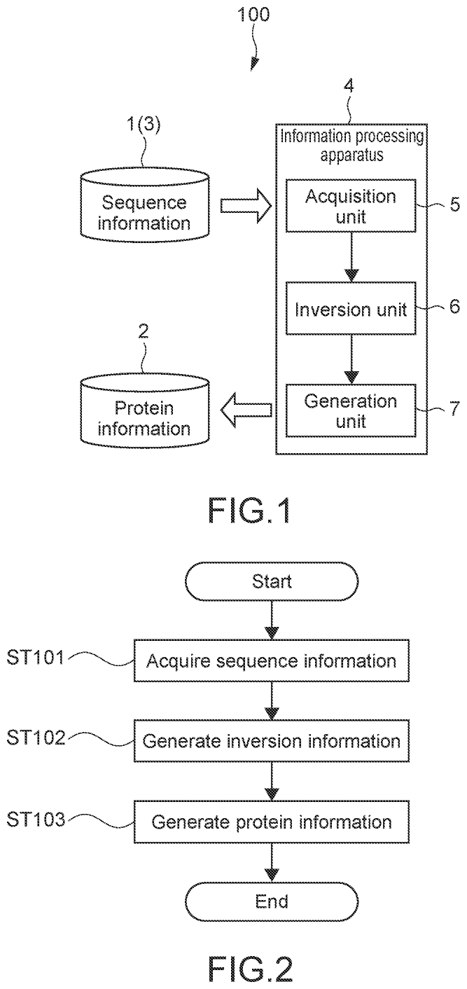

FIG. 1 is a schematic diagram showing a configuration example of a protein analysis system according to an embodiment of the present technology.

FIG. 2 is a flowchart showing an example of generating protein information by the protein analysis system.

The protein analysis system corresponds to an embodiment of an information processing system according to the present technology.

A protein analysis system 100 is capable of acquiring sequence information 1 relating to a genome sequence and generating protein information 2 on the basis of the acquired sequence information 1.

In this embodiment, as the sequence information 1 relating to a genome sequence, information relating to at least one of a sequence of amino acids, a sequence of DNA (deoxyribonucleic acid), or a sequence of RNA (ribonucleic acid) is acquired. It goes without saying that the present technology is not limited thereto, arbitrary sequence information 1 relating to a genome sequence may be acquired.

The protein information 2 includes arbitrary information relating to a protein. In this embodiment, as the protein information 2, information relating to at least one of a structure of a protein or a function of a protein is generated. In addition, arbitrary information relating to a protein may be generated.

By using this protein analysis system 100, for example, it is possible to predict the structure and function of a protein for which only the sequence of amino acids is known.

As shown in FIG. 1, the protein analysis system 100 includes a sequence information DB (database) 3 and an information processing apparatus 4.

The sequence information 1 is stored in the sequence information DB 3. For example, the sequence information 1 may be registered in the sequence information DB 3 by a user (operator) or the like. Alternatively, the sequence information 1 may be automatically collected via a network or the like.

The sequence information DB 3 includes a storage device such as an HDD and a flash memory.

In the example shown in FIG. 1, the sequence information DB 3 is constructed by a storage device external to the information processing apparatus 4. The present technology is not limited thereto, and the sequence information DB 3 may be constructed by a storage device included in the information processing apparatus 4. In this case, it functions as one embodiment of the information processing apparatus 4 including also the storage device.

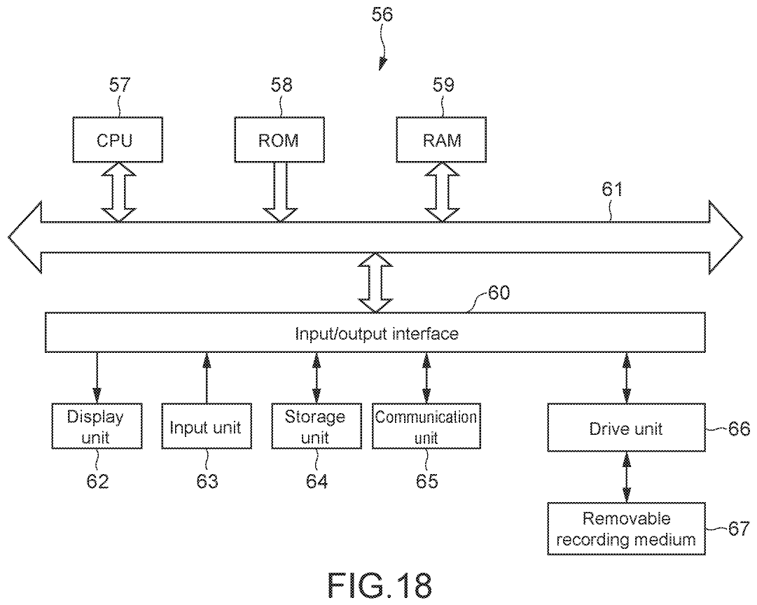

The information processing apparatus 4 includes, for example, hardware necessary for configuring a computer, such as a processor such as a CPU, a GPU, and a DSP, a memory such as a ROM and a RAM, and a storage device such as an HDD (see FIG. 18).

For example, the CPU loads a program according to the present technology recorded in the ROM or the like in advance and executes the program, thereby executing an information processing method according to the present technology.

For example, the information processing apparatus 4 can be realized by an arbitrary computer such as a PC (Personal Computer). It goes without saying that hardware such as FPGA and ASIC may be used.

In this embodiment, the CPU or the like executes a predetermined program, thereby configuring, as functional blocks, an acquisition unit 5, an inversion unit 6, and a generation unit 7. It goes without saying that in order to realize the functional blocks, dedicated hardware such as an IC (integrated circuit) may be used.

A program is installed in the information processing apparatus 4 via, for example, various recording mediums. Alternatively, a program may be installed via the Internet or the like.

The type and the like of the recording medium on which a program is recorded are not limited, and an arbitrary computer-readable recording medium may be used. For example, a computer-readable non-transitory storage medium may be used.

The acquisition unit 5 acquires the sequence information 1 relating to a genome sequence. In this embodiment, the sequence information 1 stored in the sequence information DB 3 is acquired by the acquisition unit 5.

The inversion unit 6 generates, on the basis of the sequence information 1, inversion information in which the sequence is inverted.

The generation unit 7 generates, on the basis of the inversion information, protein information 2 relating to a protein. Note that the generation of the protein information 2 based on the inversion information includes generation of the protein information 2 by an arbitrary generation method (Algorithm) Using the Inversion Information.

### [Sequence Information]

As shown in FIG. 2, the acquisition unit 5 acquires the sequence information 1 relating to a genome sequence (Step 101).

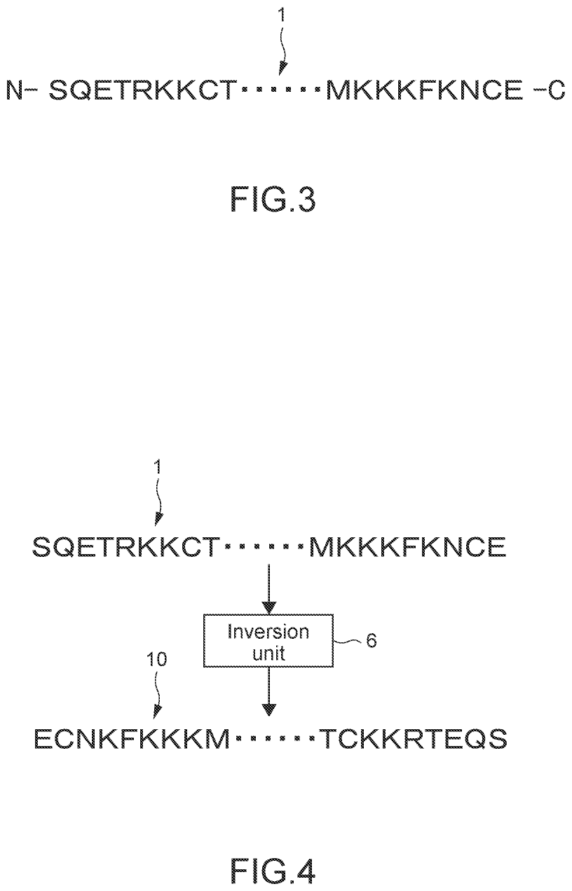

FIG. 3 is a schematic diagram showing an example of the sequence information 1.

In this embodiment, as the sequence information 1, a sequence of amino acids is acquired. For example, a character string in which a sequence of amino acids forming a protein is represented by the alphabet, as shown in FIG. 3, is acquired by the acquisition unit 5.

The structure of a protein can be expressed by a sequence of amino acid residues. However, in general, a protein having a function includes tens to thousands of amino acid residues, and it is very redundant to represent these amino acid residues by a rational formula or the like.

In this regard, in order to simply represent the sequence of amino acid residues, a method of expressing the type of an amino acid residue by one letter of the alphabet is often used. For example, a glycine residue is represented by “G” and an alanine residue is represented by “A”. In addition, each of 22 types of amino acid residues is expressed by one letter of the alphabet.

In this embodiment, such an alphabetic character string is acquired as a sequence of amino acids by the acquisition unit 5. Note that such an alphabetic character string expressing a sequence of amino acid residues is referred to as a primary structure.

In the case where a sequence of amino acid residues is expressed by the alphabet, the amino acid residues are usually described in order from the N-terminal to the C-terminal of a protein.

As shown in FIG. 3, in this embodiment, the sequence information 1 is information indicating a bonding order of amino acid residues forming a protein from the N-terminal side.

Note that the “N” and “C” described at both ends of the sequence information 1 respectively indicate positions of residues corresponding to the N-terminal and the C-terminal.

For example, the “S” described at the left end of the sequence information 1 is an alphabet indicating a serin residue. As shown in FIG. 3, the serin residue is a residue corresponding to the N-terminal.

Further, the “Q” described at the second position from the left end is an alphabet indicating a glutamine residue.

Further, the “E” described at the right end is an alphabet indicating a glutamic acid residue. As shown in FIG. 3, the glutamic acid residue is a residue corresponding to the C-terminal.

Therefore, the sequence information 1 shown in FIG. 3 indicates a sequence in which residues are lined up in the order of a serin residue, a glutamine residue, . . . , a glutamic acid residue.

In this embodiment, a sequence of amino acids expressed in this way is acquired by the acquisition unit 5.

It goes without saying that the method of expressing a sequence of amino acids is not limited to the alphabetic character string. For example, information in which a sequence of amino acids is represented by a structural formula, a rational formula, or the like may be acquired as the sequence information 1.

In the case where a sequence of DNA is acquired as the sequence information 1, for example, a base molecule is acquired.

There are four types of substances, i.e., adenine, guanine, cytosine, and thymine, as bases forming DNA. The bonding order of the four types of substances is referred to as a base sequence.

Each of the bases is often abbreviated by one letter of the alphabet. For example, adenine is represented by “A”. Similarly, guanine, cytosine, and thymine are respectively represented by “G”, “C”, and “T”.

For example, a in which a base sequence is expressed by an alphabetic character string is acquired as the sequence information 1 by the acquisition unit 5.

It goes without saying that a structural formula, a rational formula, or the like of a DNA molecule may be acquired as a

In the case where a sequence of RNA is acquired as the sequence information 1, a base molecule may be acquired.

There are four types of substances, i.e., adenine, guanine, cytosine, and uracil, as bases forming RNA.

Each of the bases is often abbreviated by one letter of the alphabet. Similarly to the case of representing a base adenine, guanine, and cytosine are respectively represented by “A”, “G”, and “C”. Further, uracil is represented by “U”.

For example, a in which a base sequence is expressed by an alphabetic character string is acquired as the sequence information 1 by the acquisition unit 5.

It goes without saying that a structural formula, a rational formula, or the like of an RNA molecule may be acquired as a

A protein is produced on the basis of a DNA sequence in a living body. Specifically, DNA is transcribed to produce RNA. RNA is translated to produce amino acids. Then, the amino acids are bonded together to produce a protein.

That is, a sequence of DNA, a sequence of RNA, and a sequence of amino acids provide pieces of information associated with each other.

In this embodiment, the sequence information 1 relating to a genome sequence is acquired by the acquisition unit 5.

The genome sequence is a term that means a base sequence DNA and a base sequence of RNA. Therefore, a sequence of DNA and a sequence of RNA are included in the sequence information 1 relating to a genome sequence.

Further, a sequence of amino acids is a sequence generated on the basis of a sequence of DNA or a sequence of RNA. Therefore, also a sequence of amino acids is included in the sequence information 1 relating to a genome sequence.

In addition, the information acquired as the sequence information 1 is not limited, and arbitrary information relating to a genome sequence may be acquired.

In the present disclosure, acquiring information includes generating the information. Therefore, the acquisition unit 5 generates the sequence information 1 in some cases.

It goes without saying that the method of generating the sequence information 1 by the acquisition unit 5 is not limited.

### [Inversion Information]

As shown in FIG. 2, the inversion unit 6 generates, on the basis of the sequence information 1, inversion information in which the sequence is inverted (Step 102).

FIG. 4 is a schematic diagram for describing inversion information.

In FIG. 4, examples of the sequence information 1 and inversion information 10 generated by the inversion unit 6 are shown.

As shown in FIG. 4, the inversion information 10 is information in which the sequence of the sequence information 1 is inverted. Specifically, information obtained by reversing the order of alphabets indicating of the sequence of an amino acid residue is generated as the inversion information 10.

For example, the “E” located at the right end of the sequence information 1 is located at the left end of the inversion information 10. Further, the “C” located at the second position from the right end of the sequence information 1 is located at the second position from the left end of the inversion information 10. Further, the “S” located at the left end of the sequence information 1 is located at the right end of the inversion information 10.

In this way, the inversion unit 6 executes processing of reversing the order of the alphabets in the sequence information 1 to generate the inversion information 10.

Therefore, the inversion information 10 is information indicating the bonding order from the C-terminal side of the sequence information 1.

### [Protein Information]

As shown in FIG. 2, the generation unit 7 generates, on the basis of the inversion information 10, the protein information 2 relating to a protein (Step 103).

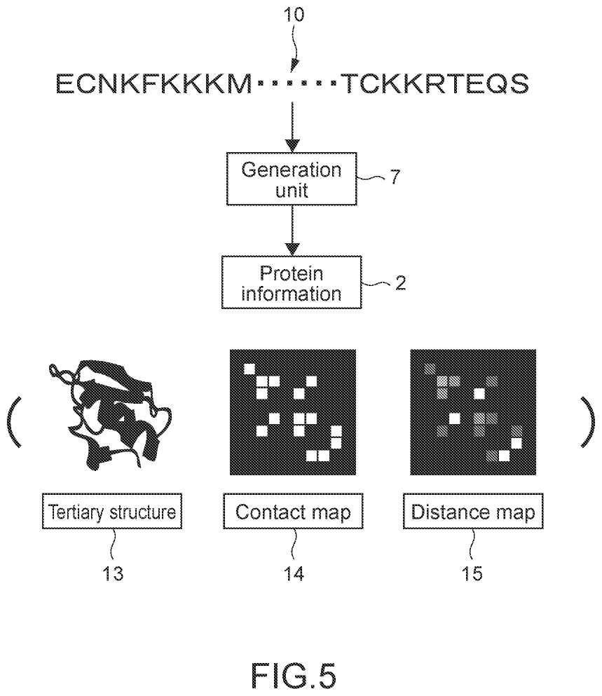

FIG. 5 is a schematic diagram for describing the protein information 2.

As shown in FIG. 5, the generation unit 7 generates the protein information 2 on the basis of the inversion information 10 generated by the inversion unit 6.

FIG. 5 shows a schematic diagram representing a tertiary structure 13, a contact map 14, and a distance map 15 as an example of the generated protein information 2.

When a protein is generated by bonding amino acids together, the protein is folded in accordance with the sequence of amino acids and has a unique three-dimensional structure. Such a three-dimensional structure taken by a protein is referred to as the tertiary structure 13.

Note that the folding of a protein is called folding in some cases.

A sequence of amino acids (primary structure) provides information indicating a simple bonding order of amino acids forming a protein. Meanwhile, the tertiary structure 13 contains information such as how the protein is folded and what shape it has as a whole.

The tertiary structure 13 can be defined by, for example, three-dimensional coordinates of each of amino acid residue.

For example, relative coordinates of each of amino acid residues are defined with reference to the coordinates of one of the amino acid residues forming a protein. It goes without saying that the method for defining the three-dimensional coordinates of each of amino acid residue is not limited, and the three-dimensional coordinates may be arbitrarily set.

For example, an arbitrary coordinate system such as a Cartesian coordinate system and a polar coordinate system may be used. Further, three-dimensional coordinates of each of atoms, molecules, functional groups, and the like forming a protein may be generated as the tertiary structure 13.

Further, as the tertiary structure 13, information other than three-dimensional coordinates may be generated. For example, information regarding a folding position of a protein, an angle of folding, or the like may be generated. In addition, arbitrary information capable of indicating a three-dimensional structure that can be taken by a protein may be used as the tertiary structure 13.

The contact map 14 is information indicating a bond between amino acid residues forming a protein. That is, the contact map 14 is a map indicating the presence or absence of a bond between residues. For example, as the contact map 14, a two-dimensional square map is used.

A residue number is assigned to each of the vertical axis and the horizontal axis of the map. The residue number is a number representing what number an amino acid residue is bonded in a protein.

For example, in a protein having the sequence information 1 as shown in FIG. 3, the “S” located at the left end of the sequence, i.e., a serin residue, corresponds to the residue of the residue number 1. Further, the “Q” located at the second position from the left end, i.e., a glutamine residue, corresponds to the residue of the residue number 2. In this way, residue numbers are assigned in order from the leftmost residue in the sequence information 1.

In the case where two amino acid residues are bonded together, points on the map corresponding to the two residue numbers are represented in white. In the case where they are not bonded together, they are represented in black.

For example, in the case where the amino acid residue of the residue number 80 and the amino acid residue of the residue number 150 are bonded together, the point on the map where the 80th position on the vertical axis and the 150th position on the horizontal axis intersect is displayed in white.

In this case, also the point on the map where the 150th position on the vertical axis and the 80th position on the horizontal axis intersect is displayed in white, similarly. Therefore, the contact map 14 is a map symmetrical with respect to the diagonal line (a set of points in which residue numbers in the vertical axis an the horizontal axis match).

Note that the colors and the like used to express the bonding state are not limited. For example, the bonding state may be expressed in colors other than white and black.

The contact map 14 is a map showing the bonding state between residues for all combinations of residues.

With the contact map 14, it is possible to estimate the three-dimensional structure of a protein, such as how the protein is folded.

For example, assumption is made that information indicating that the 80th residue and the 150th residue are bonded together is acquired from the contact map 14. However, since the 80th residue and the 150th residue are located at distant positions in the sequence, they are not bonded by a peptide bond.

From this, it is conceivable that the protein is folded at a position between the 80th residue and the 150th residue and the residues are bonded together by an ionic bond or the like. In this way, it is possible to estimate, from the contact map 14, a three-dimensional structure such as how the protein is folded.

The contact map 14 corresponds to an embodiment of protein information according to the present technology.

The distance map 15 is a map showing the distance between amino acid residues. For example, as the distance map 15, a two-dimensional square map is used similarly to the contact map 14.

Further, similarly to the contact map 14, residue numbers are assigned to the vertical axis and the horizontal axis of the map.

For example, in the distance map 15, the distance between two amino acid residues is expressed in monochrome brightness.

The distance between amino acid residues is expressed in a monochrome color with higher brightness as the distance is shorter. For example, a state in which the distance between amino acid residues is short is expressed in a color close to white. Meanwhile, for example, a state in which the distance between amino acid residues is long is expressed in a color close to black.

Note that the method of expressing the distance between amino acid residues is not limited. For example, the distance may be expressed by the brightness, saturation, hue, and the like of a color.

The distance map 15 is a map symmetrical with respect to the diagonal line, similarly to the contact map 14.

The distance map 15 is a map showing the distance between amino acid residues for all combinations of residues.

Similarly to the contact map 14, with the distance map 15, it is possible to estimate the three-dimensional structure of a protein.

The distance map 15 corresponds to an embodiment of protein information according to the present technology.

In this embodiment, as the protein information 2, at least one of a structure of a protein or a function of a protein is generated.

The structure of a protein represents the arrangement or relationship of partial elements forming a protein. For example, information such as three-dimensional coordinates of a residue and a position and angle of folding of a protein as described above corresponds to the structure of a protein. Further, as the structure of a protein, coordinates where each of bonds such as hydrogen bonds and ionic bonds is located may be generated. In addition, the information to be generated as the structure of a protein is not limited.

The tertiary structure 13, the contact map 14, and the distance map 15 shown in FIG. 5 are included in information regarding a structure of a protein.

The function of a protein represents, for example, a function that a protein has in a living body.

For example, a contractile function for moving the body, a transport function for transporting nutrients and oxygen, or an immune function corresponds to the function of a protein. In addition, the information to be generated as the function of a protein is not limited.

Note that the function of a protein appears due to the structure of the protein in some cases. For example, it is known that the protein of an antibody having an immune function has a Y-shape and traps a foreign substance in the two arm portions thereof. In this way, the function of a protein is revealed along with the generation of the structure of the protein in some cases.

In addition, the protein information 2 to be generated by the protein analysis system 100 is not limited, and arbitrary information relating to a protein may be generated.

The protein information 2 generated by the generation unit 7 is stored in, for example, a storage device in the information processing apparatus 4. Further, for example, a database may be constructed in a storage device external to the information processing apparatus 4 and protein information may be output to the database. In addition, the output method, storage method, and the like of the generated protein information 2 are not limited.

Although a sequence of amino acids, the inversion of the sequence of amino acids, generation of the protein information 2 based on the inverted sequence of amino acids, and the like have been described with reference to FIG. 1 to FIG. 5, a series of processes can be executed without being limited to the case where the sequence information 1 is a sequence of amino acids.

For example, in the case where the sequence information 1 is a sequence of DNA, a base sequence of DNA expressed as “GAATTC” is inverted by the inversion unit 6 in similar processing. Further, the generation unit 7 generates the protein information 2 on the basis of the inverted character string.

Further, also in the case where the sequence information 1 is a sequence of RNA, inversion by the inversion unit 6 and generation by the generation unit 7 are executed in similar processing.

Further, in the case where the sequence information 1 is a sequence of DNA or a sequence of RNA, the series of processes may include a process corresponding to translation of a base sequence.

In this case, for example, the information processing apparatus 4 includes a translation unit (not shown), and the translation unit executes a process corresponding to translation of a base sequence. For example, in the case where the sequence information 1 is a sequence of DNA, a process of replacing thymine (T) in the base sequence of DNA with uracil (U) to generate a base sequence of RNA is executed. Further, a process of translating a three-base sequence of RNA into one amino acid on the basis of the genetic code table to generate a sequence of amino acids may be executed.

On the basis of the sequence of amino acids generated in this way, the generation of the inversion information 10 by the inversion unit 6 and the generation of the protein information 2 by the generation unit 7 are executed.

It goes without saying that the protein information 2 may be directly generated without including a process corresponding to translation. That is, the protein information 2 may be directly generated from a sequence of DNA or a sequence of RNA without going through the generation of a sequence of amino acids.

### First Embodiment

A first embodiment will be described for details of the protein analysis system 100 shown in FIG. 1.

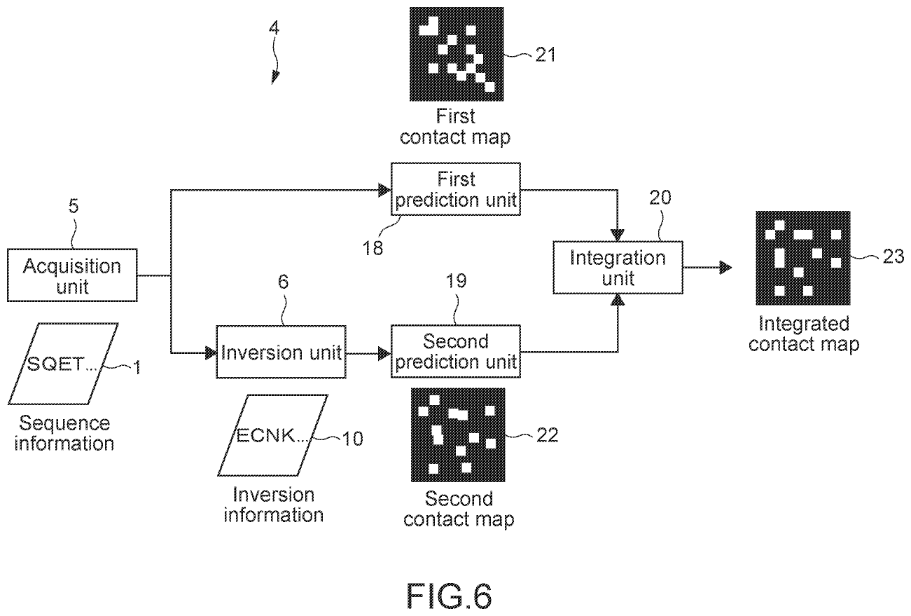

FIG. 6 is a block diagram showing a functional configuration example of the information processing apparatus 4 according to a first embodiment.

As shown in FIG. 6, the information processing apparatus 4 includes the acquisition unit 5, the inversion unit 6, a first prediction unit 18, a second prediction unit 19, and an integration unit 20.

The respective functional blocks shown in FIG. 6 are realized by a processor executing an application program or the like according to the present technology. It goes without saying that in order to realize the functional blocks, dedicated hardware such as an IC (integrated circuit) may be used.

As shown in FIG. 6, in this embodiment, the first prediction unit 18 predicts a first contact map 21. Further, the second prediction unit 19 predicts a second contact map 22. Further, the integration unit 20 integrates the first contact map 21 and the second contact map 22, thereby generating the contact map 14 as the final protein information 2.

The acquisition unit 5 acquires the sequence information 1 relating to a genome sequence. In this embodiment, as the sequence information 1, an alphabetic character string representing a sequence of amino acids is acquired.

The inversion unit 6 generates, on the basis of the sequence information 1, the inversion information 10 in which the sequence is inverted.

The first prediction unit 18 predicts first protein information on the basis of the sequence information 1.

In this embodiment, as the first protein information, the first contact map 21 is predicted.

In order to predict the first contact map 21, an arbitrary algorithm may be used. That is, an arbitrary prediction process using the sequence information 1 as an input and the first contact map 21 as an output may be executed.

The algorithm for prediction can be created, for example, in consideration of a known method for predicting a structure of a protein. For example, in the case where a method of estimating a partial structure or function of a protein from the sequence information 1 is established, a process corresponding to the procedure for estimation is incorporated into the algorithm. Specifically, a process such as numerical calculation for estimation is incorporated into the algorithm.

For example, an algorithm may be created in consideration of a known method for predicting a structure of a protein such as X-ray crystallography and nuclear magnetic resonance.

In this embodiment, a machine learning algorithm is used to predict the first contact map 21. That is, the first prediction unit 18 executes machine learning using the sequence information 1 as an input to predict the first contact map 21.

The second prediction unit 19 predicts second protein information on the basis of the inversion information 10.

In this embodiment, as the second protein information, the second contact map 22 is predicted.

As shown in FIG. 6, in this embodiment, the inversion information 10 generated by the inversion unit 6 is output to the second prediction unit 19. The second prediction unit 19 predicts the second contact map 22 on the basis of the inversion information 10.

In order to predict the second contact map 22, an arbitrary algorithm may be used. That is, an arbitrary prediction process using the inversion information 10 as an input and the second contact map 22 as an output may be executed.

In this embodiment, a machine learning algorithm is used to predict the second contact map 22. That is, the second prediction unit 19 executes machine learning using the inversion information 10 as an input to predict the second contact map 21.

Note that in order to execute each of the prediction of the first contact map 21 by the first prediction unit 18 and the prediction of the second contact map 22 by the second prediction unit 19, the same algorithm may be used or different algorithms may be used.

The integration unit 20 integrates the first contact map 21 and the second contact map 22 to generate an integrated contact map 23.

As shown in FIG. 6, the first contact map 21 predicted by the first prediction unit 18 is output to the integration unit 20. Similarly, the second contact map 22 predicted by the second prediction unit 19 is output to the integration unit 20. When the integration unit 20 receives the first contact map 21 and the second contact map 22, the first contact map 21 and the second contact map 22 are integrated to generate the integrated contact map 23.

In order to generate the integrated contact map 23, an arbitrary algorithm may be used. That is, an arbitrary integration process using the first contact map 21 and the second contact map 22 as inputs and the integrated contact map 23 as an output may be executed.

For example, information of part of the first contact map 21 and information of part of the second contact map 22 may be integrated to generate the integrated contact map 23.

For example, assumption is made that the first contact map 21 and the second contact map 22 having residue numbers ranging from 1 to 100 are predicted. The information of the first contact map 21 having the residue numbers from 1 to 50 and the information of the second contact map 22 having the numbers from 51 to 100 may be integrated to generate the integrated contact map 23.

Note that part of the first contact map 21 or the second contact map 22 may be treated as image data to execute extraction and integration processes. Further, part of the first contact map 21 or the second contact map 22 may be treated as numerical data (e.g., data in which coordinates and numerical values representing white/black are associated with each other) to execute a process.

For example, the algorithm of the integration unit 20 can be created in consideration of a known method for predicting a structure of a protein, similarly to the algorithms of the first prediction unit 18 and the second prediction unit 19.

For example, an algorithm for integration can be created such that the integrated contact map 23 is as close to the actual contact map 14 as possible in consideration of a known method for predicting a structure of a protein.

In this embodiment, a machine learning algorithm is used to predict the integrated contact map 23. That is, the integration unit 20 executes machine learning using the first contact map 21 and the second contact map 22 as inputs to predict the integrated contact map 23.

In the example shown in FIG. 6, the information processing apparatus 4 generates the contact map 14. It goes without saying that the tertiary structure 13 or the distance map 15 as shown in FIG. 5 may be generated.

Further, for example, two or more of the tertiary structure 13, the contact map 14, and the distance map 15 may be generated as the protein information 2. In this case, the first prediction unit 18 or the second prediction unit 19 may predict a plurality of pieces of information, of the tertiary structure 13, the contact map 14, and the distance map 15.

It goes without saying that the pieces of information to be predicted by the first prediction unit 18, the second prediction unit 19, and the integration unit 20 are not limited to the tertiary structure 13, the contact map 14, and the distance map 15, and arbitrary information relating to a protein may be predicted.

Further, the first prediction unit 18 may include a plurality of first prediction units that predicts first protein information on the basis of the sequence information 1. Similarly, the second prediction unit 19 may include a plurality of second prediction units that predicts second protein information on the basis of the inversion information 10.

Then, a plurality of pieces of first protein information and a plurality of pieces of second protein information may be integrated to generate the final protein information 2.

In the description with reference to FIG. 6, the operation of each functional block has been described in the order of the acquisition unit 5, the inversion unit 6, the first prediction unit 18, the second prediction unit 19, and the integration unit 20. However, the order of processing relating to a process of generating the integrated contact map 23 by the information processing apparatus 4 is not limited to this order. The order of processing by each functional block is not limited, and the processing may be executed in arbitrary order as long as a series of processes is possible.

In this embodiment, the first prediction unit 18, the second prediction unit 19, and the integration unit 20 realize the generation unit 7 shown in FIG. 1.

Further, a series of operations in which the first prediction unit 18 predicts the first contact map 21, the second prediction unit 19 predicts the second contact map 22, and the integration unit 20 predicts the integrated contact map 23 corresponds to the generation of the protein information 2 by the generation unit 7.

Thus, the generation of the protein information 2 by the generation unit 7 includes a partial process for generating the protein information 2, such as the prediction of the first contact map 21 by the first prediction unit 18, the prediction of the second contact map 22 by the second prediction unit 19, and the prediction of the integrated contact map 23 by the integration unit 20.

It goes without saying that in order to generate the protein information 2, an arbitrary process other than prediction and integration may be executed.

### [Machine Learning Model]

In this embodiment, each of the first prediction unit 18, the second prediction unit 19, and the integration unit 20 includes a machine learning model, and the machine learning executes prediction and integration.

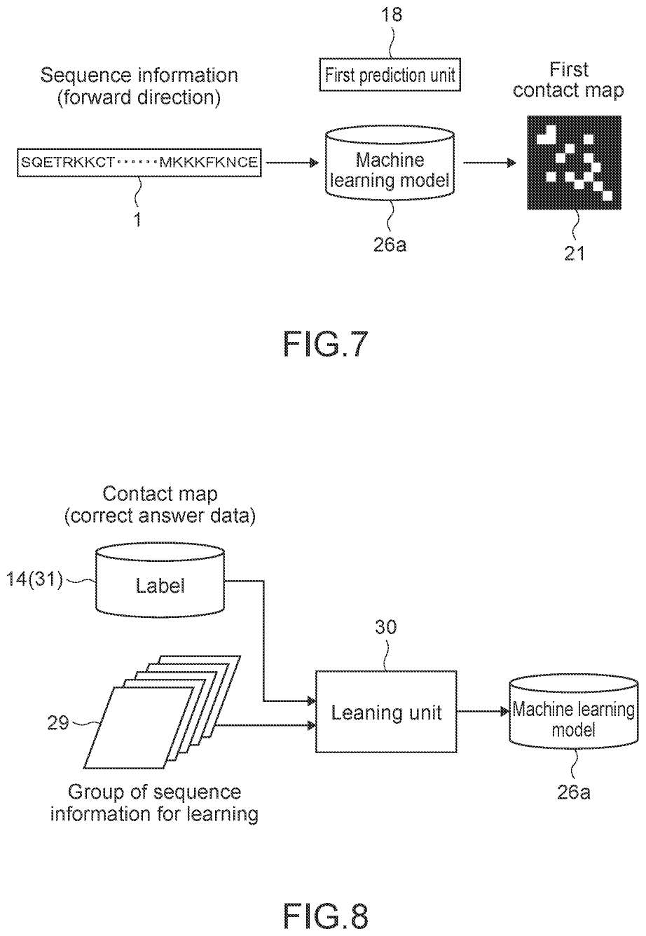

FIG. 7 is a schematic diagram showing an example of a machine learning model in the first prediction unit 18.

FIG. 8 is a schematic diagram for describing training of a machine learning model using teaching data in the first prediction unit 18.

The first prediction unit 18 executes machine learning using the sequence information 1 as an input to predict the first contact map 21.

In FIG. 7, as an example of the machine learning model, a machine learning model 26a included in the first prediction unit 18 is shown.

As shown in FIG. 7, the sequence information 1 is input to the machine learning model 26a. For example, the sequence information 1 such as a sequence of amino acids, a sequence of DNA, and a sequence of RNA is input to the machine learning model 26a.

In this embodiment, an alphabetic character string representing a sequence of amino acids is input to the machine learning model 26a.

Further, the machine learning model 26a predicts the first contact map 21.

In order to train the machine learning model 26a, teaching data in which a teacher label is associated with learning data is input to a learning unit 30. The teaching data is data for training the machine learning model that predicts a correct answer for an input.

As shown in FIG. 8, in this embodiment, as the learning data, sequence information for learning 29 is input to the learning unit 30.

Further, as the teacher label, the contact map 14 is input to the learning unit 30. The teacher label is a correct answer (correct answer data) corresponding to the sequence information for learning 29.

In this embodiment, data in which the contact map 14 (teacher label) is associated with the sequence information for learning 29 (learning data) corresponds to teaching data according to this embodiment.

For example, in the case where there is a protein for which the contact map 14 is known, the known contact map 14 is used as a teacher label. Further, the sequence information 1 relating to the protein is used as learning data. In this way, a plurality of pieces of teaching data in which the known contact map 14 and the sequence information 1 are associated with each other is prepared and is used for learning.

For example, a teaching data DB (database) is configured to store teaching data.

A plurality of pieces of teaching data is stored in the teaching data DB. That is, a plurality of pieces of data in which the contact map 14 is associated with the sequence information for learning 29 is stored.

Further, in the example shown in FIG. 8, a teacher label is stored in a label DB 31. The label DB 31 is constructed in, for example, the teaching data DB.

The configuration and method of storing teaching data (learning data and a teacher label) are not limited. For example, the teaching data DB and the label DB 31 may be included in the information processing apparatus 4, and the information processing apparatus 4 may execute training of the machine learning model 26a. It goes without saying that the teaching data DB and the label DB 31 may be configured outside the information processing apparatus 4. In addition, an arbitrary configuration and an arbitrary method may be adopted.

As shown in FIG. 8, learning data and a teacher label are associated with each other and input to the learning unit 30 as teaching data.

The learning unit 30 uses the teaching data and executes learning on the basis of a machine learning algorithm. By the learning, a parameter (coefficient) for calculating a correct answer (teacher label) is updated and is generated as a learned parameter. A program in which the generated learned parameter is incorporated is generated as the machine learning model 26a.

In this embodiment, the first prediction unit 18 includes the machine learning model 26a trained on the basis of an error between the first contact map 21 and correct answer data. That is, the machine learning model 26a is trained on the basis of an error between the predicted first contact map 21 and correct answer data. Such a learning method is referred to as an error backpropagation method.

The error backpropagation method is a learning method commonly used for training a neural network. The neural network is originally a model that imitates a human brain neural circuit and has a layered structure including an input layer, an intermediate layer (hidden layer), and an output layer. A neural network having a larger number of intermediate layers is particularly called a deep neural network, and the deep learning technology for training this is known as a model capable of learning various patterns hidden in large amounts of data. An error backpropagation method is one of such learning methods and is often used for training a convolutional neural network (CNN), which is used to recognize an image and video, for example.

Further, as a hardware structure for realizing such machine learning, a neurochip/neuromorphic chip incorporating the concept of a neural network can be used.

The error backpropagation method is a learning method that adjusts, on the basis of an error between an output and correct answer data, a parameter of a machine learning model such that the error is reduced.

It goes without saying that the algorithm for training the machine learning model 26a is not limited and an arbitrary machine learning algorithm may be used.

The machine learning model 26a generated by the learning unit 30 is incorporated into the first prediction unit 18. Then, the first prediction unit 18 predicts the first contact map 21.

The second prediction unit 19 executes machine learning using the inversion information 10 as an input to predict the second contact map 22.

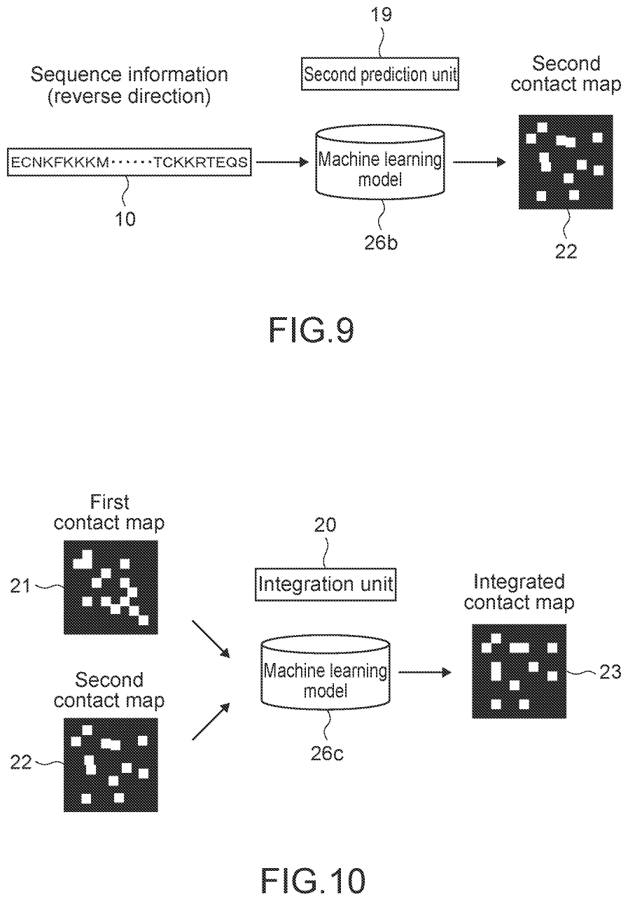

FIG. 9 is a schematic diagram showing an example of a machine learning model in the second prediction unit 19.

In FIG. 9, as an example of a machine learning model, a machine learning model 26b included in the second prediction unit 19 is shown.

As shown in FIG. 9, the inversion information 10 is input to the machine learning model 26b. In this embodiment, a character string obtained by reversing the order of the alphabetic character string representing a sequence of amino acids is input as the inversion information 10. When the inversion information 10 is input, the machine learning model 26b predicts the second contact map 22.

Similarly to the machine learning model 26a, the machine learning model 26b can be trained by an arbitrary machine learning algorithm.

For example, as in FIG. 8, inversion information for learning is input to the learning unit as learning data. Further, the contact map 14 is input to the learning unit as correct answer data.

For example, the inversion information for learning is generated by inverting the sequence information for learning 29. For example, the sequence information for learning 29 may be input to the inversion unit 6 and the inversion unit 6 may generate inversion information for learning.

It goes without saying that inversion information for learning may be prepared in advance and stored in the teaching data DB or the like.

As correct answer data, a teacher label associated with the sequence information for learning 29 can be used.

The learning unit executes learning by an error backpropagation method similarly to the machine learning model 26a to generate the machine learning model 26b. That is, the machine learning model 26b is trained on the basis of an error between the predicted second contact map 22 and correct answer data.

It goes without saying that as the method of training the machine learning model 26b, an arbitrary method (machine learning algorithm) may be adopted.

The machine learning model 26b generated by the learning unit is incorporated into the second prediction unit 19. Then, the second prediction unit 19 predicts the second contact map 22.

Note that the learning unit 30 shown in FIG. 8 may be included in the information processing apparatus 4 and the information processing apparatus 4 may execute training of the machine learning model 26a.

Similarly, the learning unit to be used for training the machine learning model 26b may be included in the information processing apparatus 4 and the information processing apparatus 4 may execute training of the machine learning model 26b.

Meanwhile, the learning unit 30 may be configured outside the information processing apparatus 4. That is, the learning of the learning unit 30 may be executed outside the information processing apparatus 4 in advance and only the trained machine learning model a may be incorporated into the first prediction unit 18.

Similarly, the learning unit to be used for training the machine learning model 26b may be configured outside the information processing apparatus 4. That is, the learning of the learning unit may be executed outside the information processing apparatus 4 in advance and only the trained machine learning model b may be incorporated into the second prediction unit 19.

In addition, the specific configurations of the learning unit 30 and the learning unit for training the machine learning model b are not limited.

The machine learning model 26a corresponds to an embodiment of a first machine learning model according to the present technology.

Further, the machine learning model 26b corresponds to an embodiment of a second machine learning model according to the present technology.

Further, the error backpropagation method corresponds to an embodiment of training based on an error between protein information and correct answer data according to the present technology.

FIG. 10 is a schematic diagram showing an example of a machine learning model in the integration unit 20.

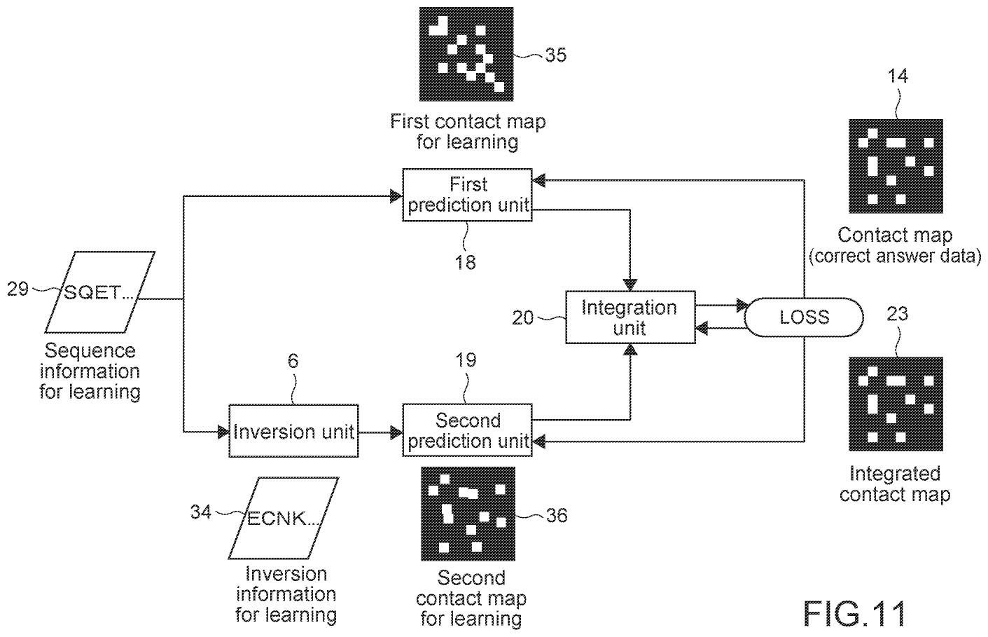

FIG. 11 is a schematic diagram for describing training of the machine learning model in the integration unit 20.

In this embodiment, the integration unit 20 includes a machine learning model 26c. Then, the integration unit 20 executes machine learning using the first contact map 21 and the second contact map 22 as inputs to predict the integrated contact map 23.

As shown in FIG. 10, the first contact map 21 predicted by the first prediction unit 18 and the second contact map 22 predicted by the second prediction unit 19 are input to the machine learning model 26c. Then, the machine learning is executed to predict the integrated contact map 23.

In the present disclosure, outputting information by machine learning using two pieces of information as inputs is included in integrating the two pieces of information to generate information.

As shown in FIG. 11, for example, the machine learning model 26c can be trained by an error backpropagation method.

Specifically, the machine learning model for integration 26c can be trained on the basis of an error between the integrated contact map 23 predicted using a first contact map for learning and a second contact map for learning as inputs and correct answer data.

Note that in FIG. 11, the training of the machine learning model 26c is illustrated as a process on the integration unit 20.

First, the sequence information for learning 29 associated with the contact map 14 as correct answer data is prepared. That is, teaching data in which the sequence information for learning 29 and the contact map 14 (correct answer data) are associated with each other is prepared.

The first contact map 21 predicted by the first prediction unit 18 using the sequence information for learning 29 as an input is used as a first contact map for learning 35.

Further, the second contact map 22 predicted by the second prediction unit 19 using the inversion information generated on the basis of the sequence information for learning 29 as an input is used as a second contact map for learning 36.

As shown in FIG. 11, inversion information for learning 34 can be generated by the inversion unit 6. It goes without saying that the present technology is not limited thereto.

The integrated contact map 23 is predicted by the integration unit 20 using the first contact map for learning 35 and the second contact map for learning 36 as inputs. The machine learning model for integration 26c is trained on the basis of an error between the predicted integrated contact map 23 and correct answer data (LOSS).

Note that the correct answer data is the contact map 14 corresponding to the sequence information for learning 1.

The machine learning model 26c generated by the learning unit 30 is incorporated into the integration unit 20. Then, the integration unit 20 predicts the integrated contact map 23.

Note that the information processing apparatus 4 may execute training of the machine learning model 26c. Alternatively, the machine learning model 26c may be trained outside the information processing apparatus 4. In addition, the specific configuration of the learning unit for training the machine learning model 26c, the learning method, and the like are not limited.

The first contact map for learning 35 corresponds to an embodiment of first protein information for learning according to the present technology.

Further, the second contact map for learning 36 corresponds to an embodiment of second protein information for learning according to the present technology.

Further, the machine learning model 26c corresponds to an embodiment of a machine learning model for integration according to the present technology.

### [Re-Training by Prediction Unit]

As shown in FIG. 11, in this embodiment, the machine learning model 26a is re-trained on the basis of an error between the integrated contact map 23 predicted by the integration unit 20 using the first contact map for learning 35 and the second contact map for learning 36 as inputs and correct answer data (LOSS).

Similarly, the machine learning model 26b is re-trained on the basis of an error between the integrated contact map 23 predicted by the integration unit 20 using the first contact map for learning 35 and the second contact map for learning 36 as inputs and correct answer data (LOSS).

That is, re-training of the machine learning model 26a and the machine learning model 26b by an error backpropagation method is executed.

As described above, in the information processing apparatus 4 according to this embodiment, the acquisition unit 5 acquires the sequence information 1 relating to a genome sequence. Further, the inversion unit 6 generates, on the basis of the sequence information 1, the inversion information 10 in which the sequence is inverted. Further, the generation unit 7 generates, on the basis of the inversion information 10, the protein information 2 relating to a protein. As a result, it is possible to predict information relating to a protein with high accuracy.

A problem of existing methods in prediction of the protein information 2 will be described.

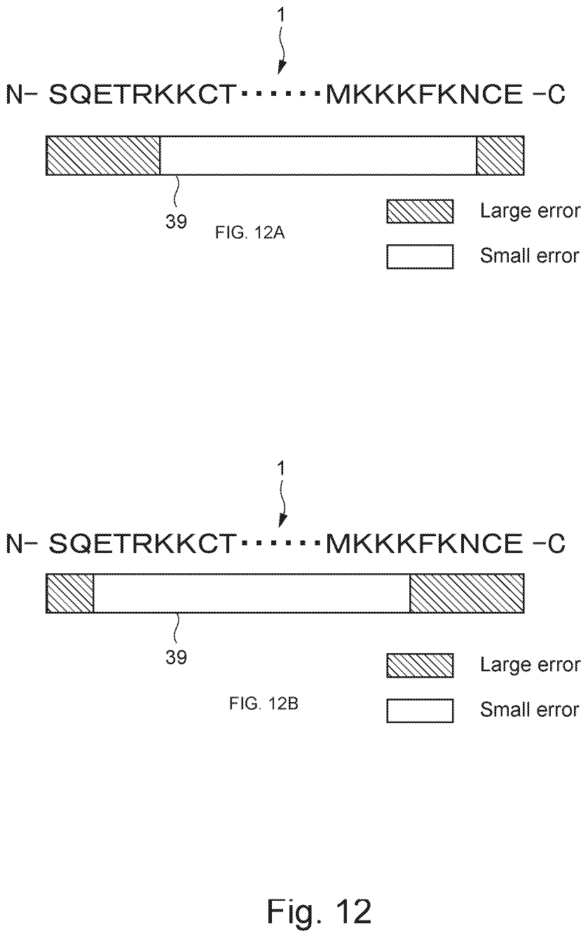

FIG. 12 is a schematic diagram for describing an error of the protein information 2.

In Parts A and B of FIG. 12, an example of an error map indicating an error of the protein information 2 predicted from the sequence information 1 by an existing method is illustrated.

An error map 39 illustrated in Parts A and B of FIG. 12 is a map representing an error in the three-dimensional coordinates of residues. Specifically, a difference in Euclidean distance between three-dimensional coordinates of residues predicted by an existing method and three-dimensional coordinates of actual residues is shown.

In the error map 39 shown in Parts A and B of FIG. 12, residue numbers are assigned from the left side to the right side in the horizontal axis. For example, a range of residue numbers having a larger error is indicated by a hatched pattern. Note that the error can be defined using a predetermined threshold value or the like.

The side having a smaller residue number (N-terminal side) corresponds to the left side of the error map 39. Further, the side having a larger residue number (C-terminal side) corresponds to the right side of the error map 39.

Therefore, for example, in the case where amino acid residues forming a protein have residue numbers from 1 to 100, the residue number 1 corresponds to the left end of the error map 39 and the residue number 100 corresponds to the right end.

The present inventor has newly found that in the prediction results by an existing method, large error portions (large errors) are unevenly distributed near both ends of the error map 39, as shown in Parts A and B of FIG. 12.

As shown in Part A of FIG. 12, large errors concentrate in a wider range on the N-terminal side in some cases. Further, as shown in Part B of FIG. 12, larges errors concentrate in a wider range on the C-terminal side in some cases.

The uneven distribution of large errors as shown in Parts A and B of FIG. 12 is considered to occur due to the time series of prediction. That is, in an existing method, prediction of the protein information 2 is processed in ascending order of residue numbers.

Therefore, it is conceivable that an error is large at the start of prediction because there is little information of an amino acid residue to be processed. As a result, it is conceivable that a phenomenon in which many errors are found near the beginning of the amino acid residues as illustrated in Part A of FIG. 12 occurs.

Further, it is conceivable that prediction errors are accumulated toward the terminal side of residues because prediction of the protein information 2 is processed in ascending order of residue numbers. As a result, it is conceivable that a phenomenon in which many errors are found near the end of the amino acid residues as shown in Part B of FIG. 12 occurs.

It is conceivable that the primary structure of a protein (sequence of amino acid residues) is responsible for whether the uneven distribution of large errors as shown in Part A of FIG. 12 or the uneven distribution of large errors as shown in Part B of FIG. 12 is provided. In each of the cases, in the predictions results by an existing method, large error portions are often unevenly distributed near both ends of the error map 39.

In this embodiment, the integration unit 20 integrates the first contact map 21 predicted on the basis of the sequence information 1 and the second contact map 22 predicted on the basis of the inversion information 10 to generate the protein information 2.

Therefore, portions of each of the first contact map 21 and the second contact map 22 with high prediction accuracy can be extracted and integrated. That is, it is possible to generate the integrated contact map 23 with fewer errors than both the first contact map 21 and the second contact map 22, the integrated contact map 23 being kind of the “best of both worlds” of the first contact map 21 and the second contact map 22.

For example, in the case where the protein information 2 to be predicted is three-dimensional coordinates, information of portions (residue numbers) with fewer errors of the three-dimensional coordinates predicted from the sequence information 1 and the three-dimensional coordinates predicted from the inversion information 10 can be integrated.

As a result, it is possible to suppress the uneven distribution of errors near both ends of the sequence of amino acid residues as shown in Parts A and B of FIG. 12, and it is possible to predict information relating to a protein with high accuracy.

Further, in this embodiment, a machine learning algorithm is used in the prediction by the first prediction unit 18 and the second prediction unit 19. Further, a machine learning algorithm is used also in the integration of the pieces of protein information 2 by the integration unit 20.

As a result, by sufficiently training each machine learning model, it is possible to execute prediction with high accuracy.

Further, in this embodiment, the re-training by the first prediction unit 18 and the second prediction unit 19 is executed in accordance with the training by the integration unit 20. As a result, it is possible to further improve the prediction accuracy.

Analysis of the three-dimensional structure of a protein is expected to be applied to various fields such as the design of medicines and the design of yeast for brewing foods.

Meanwhile, it is a difficult task to analyze the three-dimensional structure of a protein from a primary structure such as a sequence of amino acids. For example, exhaustive calculation of a three-dimensional structure requires an enormous amount of time, which is practically impossible.

By using the present technology, it is possible to predict the three-dimensional structure of a protein with high accuracy. As a result, for example, designing of medicines according to the individual, face prediction based on DNA, designing of biofuel with high accuracy, or designing of foods and crops is possible, which is expected to widely contribute to the development of technology in various fields.

### Second Embodiment

The protein analysis system 100 according to a second embodiment of the present technology will be described. In the following description, description of parts similar to the configurations and actions of the protein analysis system 100 described in the above embodiment will be omitted or simplified.

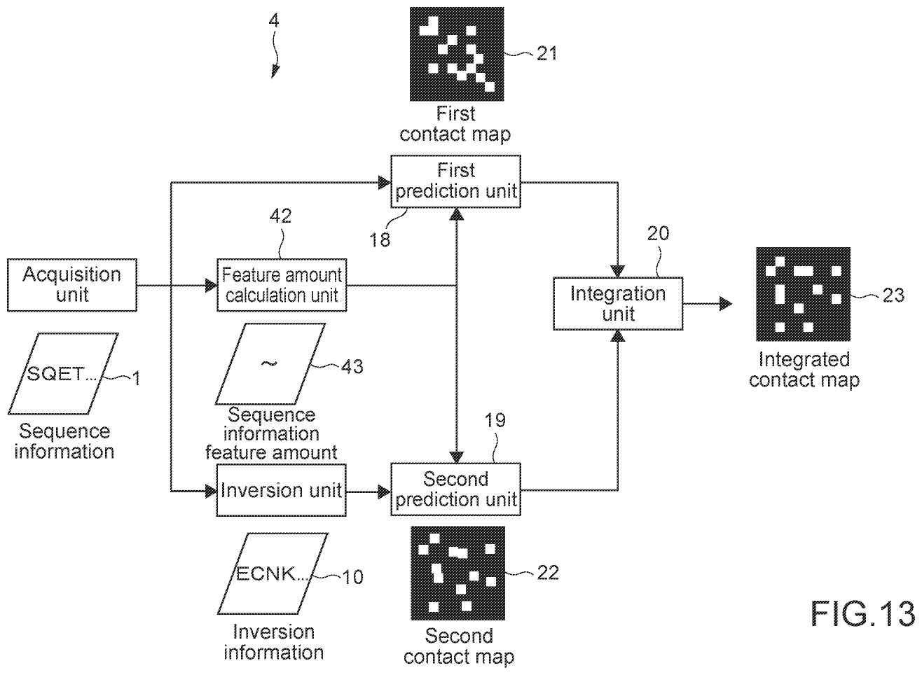

FIG. 13 is a block diagram showing a functional configuration example of the information processing apparatus 4 according to the second embodiment.

As shown in FIG. 13, the information processing apparatus 4 includes the acquisition unit 5, the inversion unit 6, a feature amount calculation unit 42, the first prediction unit 18, the second prediction unit 19, and the integration unit 20.

The respective functional blocks shown in FIG. 13 are realized by a processor executing an application program or the like according to the present technology. It goes without saying that in order to realize the functional blocks, dedicated hardware such as an IC (integrated circuit) may be used.

Since the configurations and actions of the acquisition unit 5, the inversion unit 6, and the integration unit 20 are similar to those in the first embodiment, description thereof is omitted.

In this embodiment, a feature amount indicating a feature relating to a protein is used in the prediction by the first prediction unit 18 and the second prediction unit 19. Further, training using a feature amount is executed in the first prediction unit 18, the second prediction unit 19, and the integration unit 20.

Further, similarly to the first embodiment, the contact map 14 is predicted as the protein information 2.

### [Feature Amount]

A feature amount 47 is information indicating a feature relating to a protein.

For example, a feature relating to a physical property or chemical property of a protein is used as the feature amount 47. Further, also the function or the like of a protein is used as the feature amount 47. In addition, arbitrary information indicating a feature of a protein may be used as the feature amount 47.

In this embodiment, the feature amount 47 includes at least one of a secondary structure of a protein, annotation information relating to a protein, the degree of catalyst contact of a protein, or a mutual potential between amino acid residues forming a protein.

As an example of the feature amount 47, the four feature amounts 47 described above will be described.

The secondary structure of a protein is a local three-dimensional structure of the protein. The protein is folded in accordance with the sequence of amino acids. A local three-dimensional structure is formed first in the process of folding. After that, the global folding is made to form the tertiary structure 13.

Such a local three-dimensional structure formed first at the stage before the tertiary structure 13 is formed is referred to as a secondary structure.

That is, the folding of a protein is realized in the following order; it begins with a primary structure that is a simple unfolded sequence, a secondary structure that is a local structure is formed, and the tertiary structure 13 is formed by the global folding.

As an example of the secondary structure, for example, a structure such as α-helix and β-sheet is known.

In this embodiment, the secondary structure such as α-helix and β-sheet as described above is used as the feature amount 47. It goes without saying that the secondary structure used as the feature amount 47 is not limited. For example, it is known that there is a local structure such as turns and loops as another example of the secondary structure. These secondary structures may be adopted as the feature amount 47.

The annotation information relating to a protein is metadata given (tagged) to the protein. As the metadata, typically, information relating to the protein is given. The annotation information is referred to as an annotation in some cases.

For example, as the annotation information, information relating to a structure of function of the protein is given.

As the information relating to a structure, for example, the name of the functional group of the protein is given. In addition, a molecular weight or the like of the protein may be given as the annotation information.

Further, as the information relating to a function, for example, the type of function of the protein is given. That is, annotation information such as a “contractile function”, a “transport function”, and an “immune function” is tagged.

In addition, the annotation information to be given to the protein information 2 is not limited.

The degree of catalyst contact of a protein is a value obtained by normalizing the area in which amino acid residues of the protein can be in contact with a catalyst, regardless of the size of the side chain. That is, the greater the degree of catalyst contact, the larger the area of residues in the protein in contact with the catalyst.

The degree of catalyst contact is calculated as, for example, a specific real numerical value. Note that the degree of catalyst contact is referred to as the degree of catalyst exposure or the like in some cases.

The mutual potential between amino acid residues forming a protein represents potential energy between residues.

In the case where attention is focused on two residues forming a protein, a force that depends on the distance between the residues acts on each of the residues. For example, a force acts between the residues due to the attractive force and repulsive force acting between atoms forming each of the residues.

For example, when residues get closer to each other, the repulsive force acting on each of the residues increases and the attractive force decreases. That is, a resultant force on the repulsive force side acts on each of the residues, and the respective residues try to separate.

Further, when the residues are separated from each other, the attractive force acting on each of the residues increases and the repulsive force decreases. That is, a resultant force on the attractive force side acts on each of the residues, and the respective residues try to approach.

When the distance between residues reaches a certain value, the repulsive force and the attractive force acing on each of the residues are equal to each other and the resultant force acting on each of the residues is zero. In this state, each of the residues does not try to move and is stable. In this state, the mutual potential takes the lowest value.

That is, in the case where the respective residues try to separate or approach, the mutual potential is higher than the lowest value.

In this way, the mutual potential is an index indicating whether or not each of the residues is stable.

In this embodiment, such a mutual potential is calculated as the feature amount 47.

For example, as the feature amount 47, the sum of mutual potentials between all residues forming a protein is calculated.

For example, in the case where a protein includes a residue A, a residue B, and a residue C, the mutual potential between the residue A and the residue B is calculated. Similarly, the mutual potential between the residue A and the residue C and the mutual potential between the residue B and the residue C are also calculated. The sum of the three calculated mutual potentials is used as the feature amount 47.

At least one of the secondary structure, the annotation information, the degree of catalyst contact, or the mutual potential as described above is included in the feature amount 47.

It goes without saying that the feature amount 47 is not limited to the four pieces of information described above, and arbitrary information indicating a feature relating to a protein can be used as the feature amount 47.

### [Calculation of Feature Amount]

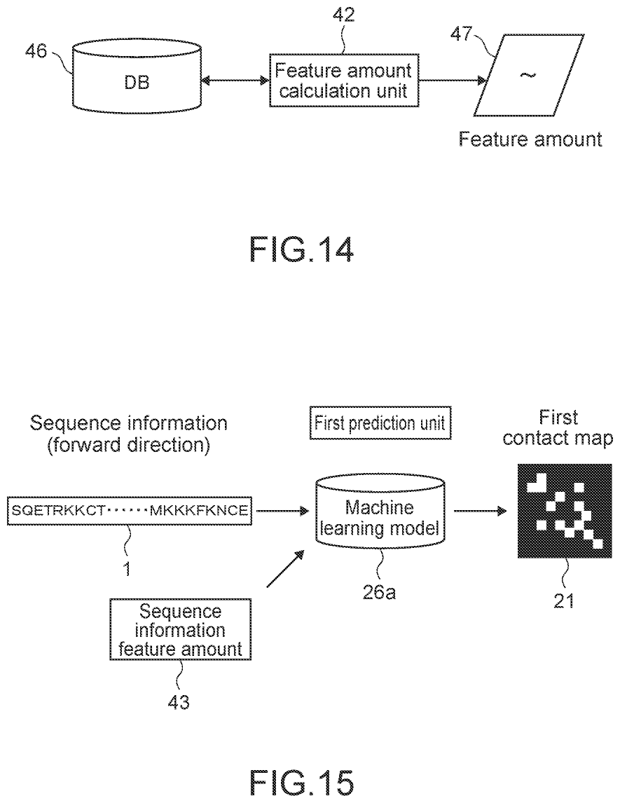

FIG. 14 is a schematic diagram for describing calculation of a feature amount.

In FIG. 14, a schematic diagram representing a database (DB) 46, the feature amount calculation unit 42, and the feature amount 47 is shown.

As shown in FIG. 14, the feature amount calculation unit 42 calculates the feature amount 47 on the basis of the sequence information 1.

Note that in FIG. 13, the feature amount calculated on the basis of the sequence information 1 is described as a sequence information feature amount 43. This is description for distinguishing from the feature amount 47 (inversion information feature amount) based on the inversion information 10, which will be described in a third embodiment. The calculation of the feature amount 47 based on the inversion information 10 will be described in the third embodiment.

The sequence information feature amount 43 corresponds to an embodiment of a first feature amount according to the present technology.

The database (DB) 46 is used to calculate a feature amount. In the database 46, data in which the sequence information 1 and the feature amount 47 are associated with each other is stored.

As shown in FIG. 14, the feature amount calculation unit 42 calculates the feature amount 47 by accessing the database 46 in which the sequence information 1 and the feature amount 47 are associated with each other.

As the database 46, an existing database that has already been constructed can be used.

An example of the method of calculating the feature amount 47 will be described.

First, the feature amount calculation unit 42 acquires the sequence information 1. For example, the sequence information 1 acquired the acquisition unit 5 is output to the feature amount calculation unit 42 and the feature amount calculation unit 42 receives the sequence information 1, thereby realizing the acquisition of the sequence information 1.

When the feature amount calculation unit 42 acquires the sequence information 1, the sequence information 1 is divided into a plurality of pieces. Hereinafter, each piece of the sequence information 1 generated by the division will be expressed as partial sequence information.

For example, in the case where the sequence information 1 is a sequence of amino acids and is an alphabetic character string representing residues, the character string is divided to generate the partial sequence information.

As an example, in the case where the original sequence information 1 is “SQETRKKCT”, two pieces of partial sequence information of “SQET” and “RKKCT” are generated by the division of the character string.

It goes without saying that the position and number of divisions of the character string are not limited to the example described above.

Further, also in the case where the sequence information 1 is a sequence of DNA or a sequence of RNA, the character string is divided similarly.

When the partial sequence information is generated, the feature amount calculation unit 42 searches the database 46 for the sequence information 1 that matches the partial sequence information.

In the database 46, data in which the sequence information 1 and the feature amount 47 are associated with each other is stored. In the case where the feature amount calculation unit 42 has found the sequence information 1 that matches the partial sequence information, the feature amount calculation unit 42 collectively extracts the sequence information 1 and the feature amount 47 associated with the sequence information 1.

Note that not the sequence information 1 that matches the partial sequence information but similar sequence information 1 may be searched for.

When the partial sequence information searches for the sequence information 1 as described above, a plurality of sets of data each including the sequence information 1 and the feature amount 47 is extracted.

The plurality of pieces of feature amount 47 acquired in this way is used for prediction.

Note that one feature amount 47 may be calculated by the feature amount calculation unit 42 on the basis of the plurality of extracted feature amount 47 and used for prediction.

The method of calculating a feature amount, which includes the division of the sequence information 1, as described above is merely an example. It goes without saying that the calculation method is not limited.

For example, the sequence information 1 that matches the sequence information 1 may be searched for without dividing the sequence information 1. In addition, as the method of calculating the feature amount 47 by the feature amount calculation unit 42, an arbitrary method can be adopted.

Note that in the database 46, for example, the feature amount 47 that is known by structural analysis of a protein that has been executed in the past is stored.

For example, there is a protein whose structure has been successfully analyzed based on the sequence information 1 by a method such as X-ray crystallography and nuclear magnetic resonance. Specifically, there is a protein for which the actual tertiary structure 13, contact map 14, or distance map 15 has been analyzed on the basis of the sequence information 1.

In such a protein, for example, the feature amount 47 of the protein has been revealed in the process of analysis in some cases. For example, the secondary structure of a protein is naturally revealed on the basis of the tertiary structure 13 of the protein.

A set of the actual sequence information 1 and the feature amount 47 that have been revealed by, for example, past research as described above is stored in the database 46.

It goes without saying that the feature amount 47 or the like acquired by past prediction may be stored in the database 46.

As shown in FIG. 13, the first prediction unit 18 predicts the first contact map 21 on the basis of the sequence information 1 and the sequence information feature amount 43.

In this embodiment, the sequence information 1 acquired by the acquisition unit 5 is output to the first prediction unit 18. Further, the sequence information feature amount 43 calculated by the feature amount calculation unit 42 is output to the first prediction unit 18. When the first prediction unit 18 receives the sequence information 1 and the sequence information feature amount 43, the first contact map 21 is predicted on the basis of the sequence information 1 and the sequence information feature amount 43.

As the prediction method, for example, prediction by a predetermined algorithm is adopted similarly to the first embodiment. Specifically, the first prediction unit 18 includes the algorithm for prediction, and a prediction process by the algorithm using the sequence information 1 and the sequence information feature amount 43 as inputs and the contact map 14 as an output is executed.

For example, the algorithm is created in consideration of a known method for predicting a structure of a protein. In this embodiment, for example, an algorithm capable of effectively using the sequence information feature amount 43 is created in order to execute prediction with high accuracy, because the sequence information feature amount 43 is input to the algorithm.

Specifically, in the case where there is a method capable of performing prediction with high accuracy by using the sequence information feature amount 43, an algorithm is created in consideration of the method.

In addition, the algorithm for prediction included in the first prediction unit 18 is not limited. For example, also in this embodiment, the first prediction unit 18 may include a machine learning algorithm. Prediction of the contact map 14 by machine learning will be described below.

Further, the prediction method by the first prediction unit 18 is not limited to prediction by an algorithm and an arbitrary prediction may be adopted.

The second prediction unit 19 predicts the second contact map 22 on the basis of the inversion information 10 and the sequence information feature amount 43.

In this embodiment, the inversion information 10 obtained by the inversion by the inversion unit 6 is output to the second prediction unit 19. Further, the sequence information feature amount 43 calculated by the feature amount calculation unit 42 is output to the second prediction unit 19. When the second prediction unit 19 receives the inversion information 10 and the sequence information feature amount 43, the second contact map 22 is predicted on the basis of the inversion information 10 and the sequence information feature amount 43.

As the prediction method by the second prediction unit 19, for example, the same method as that by the first prediction unit 18 is adopted. It goes without saying that as the prediction method by the second prediction unit 19, a method different from that prediction method by the first prediction unit 18 may be adopted.

The integration unit 20 executes an integration process based on the first contact map 21 and the second contact map 22 to generate the integrated contact map 23.

Note that the prediction using the sequence information feature amount 43 may be executed in only one of the prediction units.

For example, the first prediction unit 18 executes prediction on the basis of the sequence information 1 and the sequence information feature amount 43. Meanwhile, the second prediction unit 19 executes prediction on the basis of only the inversion information 10 (without using the sequence information feature amount 43). Such a method may be adopted as a prediction method.

Further, the order of processing relating to the process of generating the integrated contact map 23 by the information processing apparatus 4 is not limited.

For example, either the prediction by the first prediction unit 18 or the generation of the inversion information 10 by the inversion unit 6 may be executed first. Further, either the calculation of the sequence information feature amount 43 by the feature amount calculation unit 42 or the generation of the inversion information 10 by the inversion unit 6 may be executed first.

In addition, the order of processing by each functional block is not limited, and the processing may be executed in arbitrary order as long as a series of processes is possible.

### [Machine Learning Model]

Also in this embodiment, each of the first prediction unit 18, the second prediction unit 19, and the integration unit 20 includes a machine learning model, and machine learning for prediction and integration is executed.

FIG. 15 is a schematic diagram showing an example of a machine learning model in the first prediction unit 18.

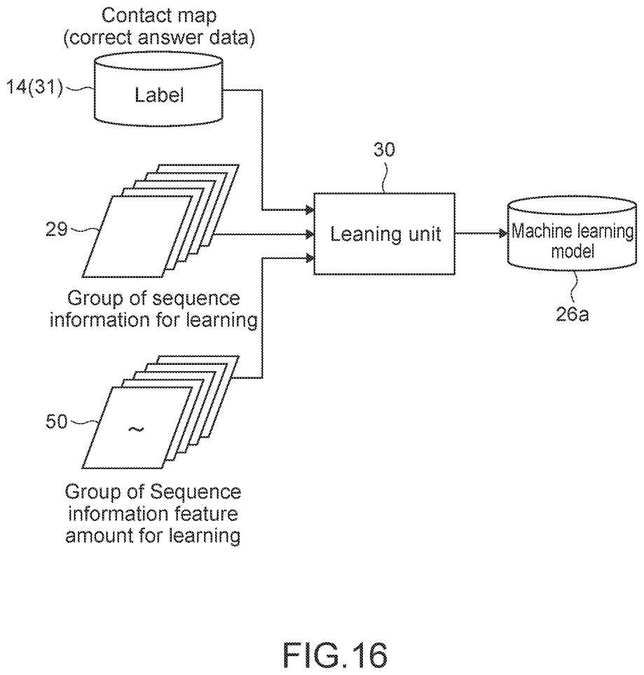

FIG. 16 is a schematic diagram for describing training of a machine learning model using teaching data in the first prediction unit 18.

Although only the sequence information 1 is used for learning of the first prediction unit 18 in the first embodiment, the sequence information 1 and the sequence information feature amount 43 are used for learning in this embodiment (second embodiment).

Further, although only the inversion information 10 is used for learning of the second prediction unit 19 in the first embodiment, the inversion information 10 and the sequence information feature amount 43 are used for learning in this embodiment.

Hereinafter, the differences described above will be mainly described, and description of content similar to that in the first embodiment will be omitted.

As shown in FIG. 15, the sequence information 1 and the sequence information feature amount 43 are input to the machine learning model 26a in the first prediction unit 18.

The machine learning model 26a predicts the first contact map 21 on the basis of the input sequence information 1 and sequence information feature amount 43.

As shown in FIG. 16, teaching data in which a teacher label is associated with learning data is input to the learning unit 30.

In this embodiment, a set of the sequence information for learning 29 and sequence information feature amount for learning corresponds to learning data.

Further, the contact map 14 corresponds to a teacher label (correct answer data).

For example, in the case where there is a protein for which the contact map 14 is known, the known contact map 14 is used as correct answer data. Further, the sequence information 1 relating to the protein is used as the sequence information for learning 29.

Further, the feature amount 47 relating to the protein is used as the sequence information feature amount for learning 50. For example, the feature amount calculation unit 42 calculates the feature amount 47 on the basis of the sequence information for learning 29, and the feature amount 47 is used as the sequence information feature amount for learning 50.

It goes without saying that the method of generating the sequence information feature amount for learning 50 is not limited and an arbitrary method may be adopted.

In this way, a plurality of pieces of teaching data in which the known contact map 14, the sequence information 1, and the sequence information feature amount 43 are associated with each other is prepared and used for learning.

The sequence information feature amount for learning 50 corresponds to an embodiment of a first feature amount for learning according to the present technology.

In this embodiment, the first prediction unit 18 includes the machine learning model 26a trained on the basis of an error between the first contact map 21 predicted using the sequence information for learning 29 associated with correct answer data and the sequence information feature amount for learning 50 calculated on the basis of the sequence information for learning 29 as inputs and the correct answer data.

That is, learning of the first prediction unit 18 is executed by an error backpropagation method on the basis of an error between the first contact map 21 and correct answer data.

It goes without saying that the learning method of the first prediction unit 18 is not limited and an arbitrary method may be adopted.

The machine learning model 26a generated by the learning unit 30 is incorporated into the first prediction unit 18. Then, the first prediction unit 18 predicts the first contact map 21.

Also in the second prediction unit 19, learning using the feature amount 47 is executed.

In this embodiment, the second prediction unit 19 includes the machine learning model 26b trained on the basis of an error between the second contact map 22 predicted using the inversion information generated on the basis of the sequence information for learning 29 and the sequence information feature amount for learning 50 calculated on the basis of the sequence information for learning 29 as inputs and the correct answer data.

Specifically, the training of the machine learning model 26b is executed by an error backpropagation method using the inversion information for learning 34 and the sequence information feature amount for learning 50 as inputs.

It goes without saying that the learning method of the second prediction unit 19 is not limited and an arbitrary method may be adopted.

Next, learning of the integration unit 20 will be described.

Also in the integration unit 20, learning is executed similarly to the first embodiment. Specifically, learning is executed by inputting the first contact map for learning 35 and the second contact map for learning 36 to the machine learning model 26c.

Note that the first contact map for learning 35 is predicted by the first prediction unit 18 on the basis of the sequence information for learning 29 and the sequence information feature amount for learning 50. Further, the second contact map for learning 36 is predicted by the second prediction unit 19 on the basis of the inversion information for learning 34 and the sequence information feature amount for learning 50.

### [Re-Training by Prediction Unit]

Similarly to the first embodiment, the machine learning model 26a is re-trained on the basis of an error between the integrated contact map 23 predicted using the first contact map for learning 35 and the second contact map for learning 36 as inputs and correct answer data.

Further, also the machine learning model 26b is re-trained on the basis of an error between the integrated contact map 23 and correct answer data.

That is, re-training of the machine learning model 26a and the machine learning model 26b is executed by an error backpropagation method.

As described above, in the information processing apparatus 4 according to this embodiment, since the sequence information feature amount 43 is used for prediction, it is possible to perform prediction with high accuracy in the first prediction unit 18 and the second prediction unit 19. Further, also the prediction results of the integrated contact map 23 generated by the integration unit 20 are highly accurate because the prediction results of the first prediction unit 18 and the second prediction unit 19 are used.

In this way, highly accurate precision is realized by using the sequence information feature amount 43.

Further, in this embodiment, since the sequence information feature amount 43 is used for also learning, a machine learning model capable of executing prediction with high accuracy is generated.

### Third Embodiment

A protein analysis system according to a third embodiment of the present technology will be described. Note that description of parts similar to the configurations and actions of the protein analysis system 100 described in the first embodiment and the second embodiment will be omitted or simplified.

In the third embodiment, the first prediction unit 18 executes prediction on the basis of the sequence information 1 and the sequence information feature amount 43.

Further, in the second embodiment, the second prediction unit 19 executes prediction and learning on the basis of the inversion information 10 and the sequence information feature amount 43.

Meanwhile, in the third embodiment, the second prediction unit 19 executes prediction and learning on the basis of the inversion information 10 and inversion information feature amount. This points is a difference between the second embodiment and the third embodiment.

[Configuration Example of Information Processing Apparatus]

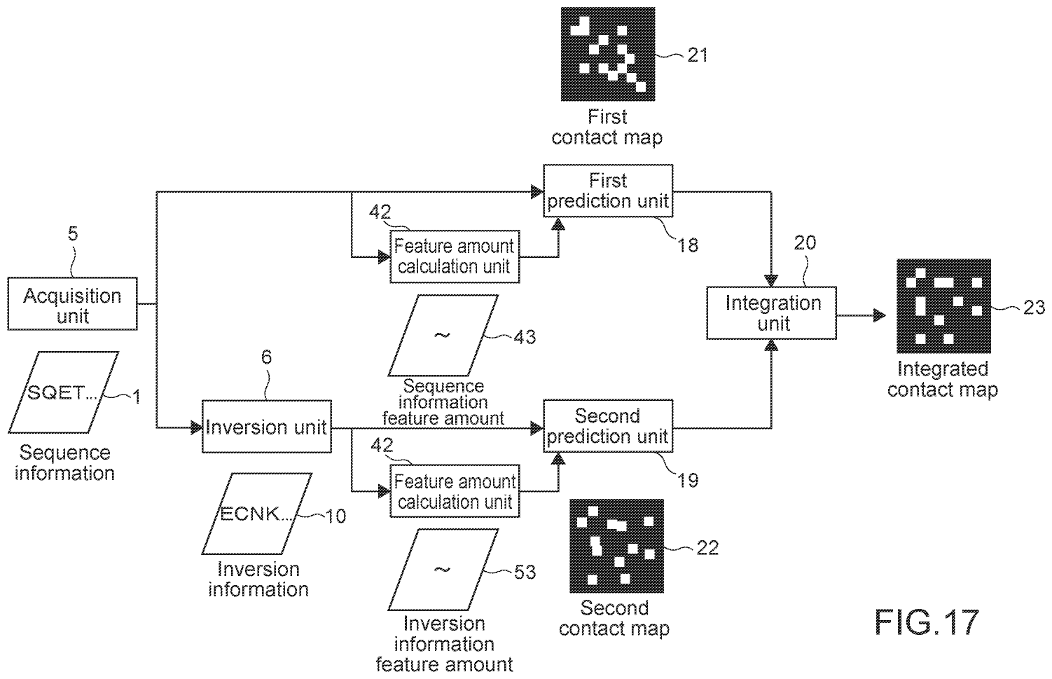

FIG. 17 is a block diagram showing a functional configuration example of the information processing apparatus 4 according to the third embodiment.

As shown in FIG. 17, the information processing apparatus 4 includes the acquisition unit 5, the inversion unit 6, the feature amount calculation unit 42, the first prediction unit 18, the second prediction unit 19, and the integration unit 20.

Since the configurations and actions of the acquisition unit 5, the inversion unit 6, the first prediction unit 18, and the integration unit 20 are similar to those in the second embodiment, description thereof is omitted.

In this embodiment, the contact map 14 is predicted as the protein information 2 similarly to the other embodiments.

As shown in FIG. 17, in this embodiment, the feature amount calculation unit 42 calculates the sequence information feature amount 43 on the basis of the sequence information 1 and inversion information feature amount 53 on the basis of the inversion information 10.

The sequence information feature amount 43 is calculated in a way similar to that in the second embodiment.

Also the inversion information feature amount 53 is calculated in a way substantially similar to that in the second embodiment. Specifically, for example, the feature amount calculation unit 42 acquires the inversion information 10, and the division of the inversion information 10, the search in a database, and the like are executed similarly o the second embodiment, thereby calculating the inversion information feature amount 53.

Note that the calculated inversion information feature amount 53 can of course be information different from the sequence information feature amount 43. This is because, for example, the partial sequence information and partial inversion information (information obtained by dividing the inversion information 10) are different pieces of information, the extraction results in the database differ, and therefore, the feature amounts 47 finally calculated also differ.

The inversion information feature amount 53 corresponds to an embodiment of a second feature amount according to the present technology.

As shown in FIG. 17, the first prediction unit 18 predicts the first contact map 21 on the basis of the sequence information 1 and the sequence information feature amount 43, similarly to the second embodiment.

Meanwhile, the second prediction unit 19 predicts the second contact map 22 on the basis of the inversion information 10 and the inversion information feature amount 53.

In this embodiment, the inversion information 10 generated by the inversion unit 6 is output to the second prediction unit 19. Further, the inversion information feature amount 53 calculated by the feature amount calculation unit 42 is output to the second prediction unit 19. When the second prediction unit 19 receives the inversion information 10 and the inversion information feature amount 53, the second contact map 22 is predicted on the basis of the inversion information 10 and the inversion information feature amount 53.

As the prediction method, for example, prediction by a predetermined algorithm is adopted similarly to the other embodiments. It goes without saying that the prediction method of the second prediction unit 19 is not limited to prediction by an algorithm and an arbitrary prediction method may be adopted.

The integration unit 20 executes an integration process based on the first contact map 21 and the second contact map 22 to generate the integrated contact map 23.

Note that the order of processing relating to the process of generating the integrated contact map 23 by the information processing apparatus 4 is not limited.

For example, either the prediction by the first prediction unit 18 or the generation of the inversion information feature amount 53 by the feature amount calculation unit 42 may be executed first.

In addition, the order of processing by each functional block is not limited, and the processing may be executed in arbitrary order as long as a series of processes is possible.

[Machine Learning Model]

Also in the third embodiment, learning by an error backpropagation method is executed similarly to the second embodiment.

The first prediction unit 18 executes learning using the sequence information for learning 29 and the sequence information feature amount for learning 50 as inputs, similarly to the second embodiment.

Meanwhile, the second prediction unit 19 includes the machine learning model 26b trained on the basis of an error between the second contact map 22 predicted using the inversion information 10 generated on the basis of the sequence information for learning 29 and an inversion information feature amount for learning calculated on the basis of the inversion information 10 as inputs and correct answer data.

That is, training of the machine learning model 26b executed by an error backpropagation method using the inversion information for learning 34 and the inversion information feature amount for learning as inputs.

It goes without saying that the learning method of the second prediction unit 19 is not limited and an arbitrary method may be adopted.

Note that, for example, the feature amount calculation unit 42 calculates the feature amount 47 on the basis of the inversion information for learning 34, and the feature amount 47 is used as the inversion information feature amount for learning.

It goes without saying that the method of generating inversion information feature amount for learning is not limited and an arbitrary method may be adopted.

The inversion information feature amount for learning corresponds to an embodiment of a second feature amount for learning according to the present technology.

Also in the integration unit 20, learning is executed similarly to the second embodiment.

The only difference from the second embodiment is that the second contact map for learning 36 is predicted on the basis of the inversion information for learning 34 and the inversion information feature amount for learning.

[Re-Training by Prediction Unit]

Also re-training by each prediction unit is similar to that in the second embodiment.

That is, re-training of the machine learning model 26a and the machine learning model 26b based on an error between the integrated contact map 23 and correct answer data is executed by an error backpropagation method.

As described above, since the sequence information feature amount 43 and the inversion information feature amount 53 are used for prediction in the information processing apparatus 4 according to this embodiment, the first prediction unit 18 and the second prediction unit 19 are capable of performing prediction with high accuracy. Further, also the prediction results of the integrated contact map 23 generated by the integration unit 20 are highly accurate because the prediction results of the first prediction unit 18 and the second prediction unit 19 are used.

In this way, highly accurate precision is realized by using the sequence information feature amount 43 and the inversion information feature amount 53.

Further, in this embodiment, since the sequence information feature amount 43 and the inversion information feature amount 53 are used for also learning, a machine learning model capable of executing prediction with high accuracy is generated.

### Other Embodiments

The present technology is not limited to the embodiments described above, and various other embodiments can be realized.

In each of the prediction units, the type of information to be input for prediction is not limited. That is, which one of the sequence information 1, the inversion information 10, the sequence information feature amount 43, and the inversion information feature amount 53 is input to the prediction unit is not limited.

Examples of combinations of the types of information to be input to the two prediction units different from those in the second embodiment and the third embodiment are as follows.

(1) The sequence information 1 and the sequence information feature amount 43 are input to the first prediction unit, and
the sequence information 1 and the inversion information feature amount 53 are input to the second prediction unit,

(2) The sequence information 1 and the inversion information feature amount 53 are input to the first prediction unit, and
the inversion information 10 and the sequence information feature amount 43 are input to the second prediction unit,

(3) The sequence information 1 and the inversion information feature amount 53 are input to the first prediction unit, and
the inversion information 10 and the inversion information feature amount 53 are input to the second prediction unit,

(4) The inversion information 10 and the sequence information feature amount 43 are input to the first prediction unit, and
the inversion information 10, and the inversion information feature amount 53 are input to the second prediction unit.

Further, it goes without saying that three or more prediction units may be configured. In this case, the combination of types of information to be input to the prediction units is also not limited.

FIG. 18 is a block diagram showing a hardware configuration example of a computer 56 capable of realizing the information processing apparatus 4.

The computer 56 includes a CPU 57, a ROM 58, a RAM 59, an input/output interface 60, and a bus 61 connecting them to each other. A display unit 62, an input unit 63, a storage unit 64, a communication unit 65, a drive unit 66, and the like are connected to the input/output interface 60.

The display unit 62 is, for example, a display device using liquid crystal, EL, or the like. The input unit 63 is, for example, a keyboard, a pointing device, a touch panel, or another operating device. In the case where the input unit 63 includes a touch panel, the touch panel can be integrated with the display unit 62.

The storage unit 64 is a non-volatile storage device, and is, for example, an HDD, a flash memory, or another solid-state memory. The drive unit 66 is a device capable of driving a removable recording medium 67 such as an optical recording medium and a magnetic recording tape.

The communication unit 65 is a modem, a router, or another communication device for communicating with another device, which is capable of connecting to a LAN, a WAN, or the like. The communication unit 65 may use either wired or wireless communication. The communication unit 65 is often used separately from the computer 56.

The information processing by the computer 56 having the hardware configuration as described above is realized by cooperation of software stored in the storage unit 64, the ROM 58, or the like, and hardware resources of the computer 56. Specifically, a program constituting software, which is stored in the ROM 58 or the like, is loaded into the RAM 59 and executed, thereby realizing the information processing method according to the present technology.

The program is installed in the computer 56 via, for example, the removable recording medium 67. Alternatively, the program may be installed in the computer 56 via a global network or the like. In addition, an arbitrary non-transitory storage medium that can be read by the computer 56 may be used.

The information processing method according to the present technology may be executed to construct the information processing apparatus 4 according to the present technology by cooperation of a plurality of computers communicably connected via a network or the like.

That is, the information processing method according to the present technology can be executed not only in a computer system including a single computer but also in a computer system in which a plurality of computers works together.

Note that in the present disclosure, the system means an aggregate of a plurality of components (such as apparatuses and modules (parts)) and it does not matter whether or not all the components are housed in the identical casing. Thus, both a plurality of apparatuses accommodated in separate housings and connected to each other through a network, and a single apparatus in which a plurality of modules is accommodated in a single housing correspond to the system.

The execution of the information processing method according to the present technology by a computer system includes, for example, a case where the prediction of the protein information 2, the calculation of the feature amount 47, and the like are executed by a single computer and a case where the respective processes are executed by different computers.

Further, the execution of the respective processes by a predetermined computer includes causing another computer to execute some or all of those processes and acquiring results thereof.

That is, the information processing method according to the present technology can be applied also to the configuration of cloud computing in which one function is shared and collaboratively processed by a plurality of apparatuses via a network.

The protein analysis system 100, the information processing apparatus 4, the information processing method, and the like described with reference to the drawings are merely embodiments, and can be arbitrarily modified without departing from the essence of the present technology. That is, another arbitrary configuration, algorithm, and the like for carrying out the present technology may be adopted.

In the present disclosure, words such as “approximately”, “substantially”, and “almost” are appropriately used to facilitate understating of the description. Meanwhile, there is no clear difference between the use and non-use of these words such as “approximately”, “substantially”, and “almost”.

That is, in the present disclosure, concepts defining a shape, a size, a positional relationship, a state, and the like, such as “central”, “middle”, “uniform”, “equal”, “the same”, “orthogonal”, “parallel”, “symmetrical”, “extended”, “axial direction”, “columnar shape”, “cylindrical shape”, “ring shape”, and “annular shape”, are concepts including “substantially central”, “substantially middle”, “substantially uniform”, “substantially equal”, “substantially the same”, “substantially orthogonal”, “substantially parallel”, “substantially symmetrical”, “substantially extended”, “substantially axial direction”, “substantially columnar shape”, “substantially cylindrical shape”, “substantially ring shape”, and “substantially annular shape”.

For example, a state included in a predetermined range (e.g., a range of ±10%) with reference to “completely central”, “completely middle”, “completely uniform”, “completely equal”, “completely the same”, “completely orthogonal”, “completely parallel”, “completely symmetrical”, “completely extended”, “completely axial direction”, “completely columnar shape”, “completely cylindrical shape”, “completely ring shape”, “completely annular shape”, and the like is also included.

Therefore, even in the case where the words such as “approximately”, “substantially”, and “almost” are not added, concepts that can be expressed by adding so-called “approximately”, “substantially”, “almost”, and the like can be included. On the contrary, the complete state is not necessarily excluded from the state expressed by adding “approximately”, “substantially”, “almost”, and the like.

In the present disclosure, expressions using “than” such as “larger than A” and “smaller than A” are expressions comprehensively including both the concept including the case where it is equivalent to A and the concept not including the case where it is equivalent to A. For example, the phrase “larger than A” is not limited to the case not including being equivalent to A and includes “A or more”. Further, the phrase “smaller than A” is not limited to “less than A” and includes “A or less”.

When implementing the present technology, specific setting and the like only need to be appropriately adopted from the concepts included in “larger than A” and “smaller than A” such that the effects described above are exhibited.

Of the feature portions according to the present technology described above, at least two feature portions can be combined. That is, the various feature portions described in the respective embodiments may be arbitrarily combined with each other without distinguishing from each other in the respective embodiments. Further, the various effects described above are merely illustrative and are not limitative, and another effect may be exhibited.

It should be noted that the present technology may also take the following configurations.
(1) An information processing apparatus, including:
   an acquisition unit that acquires sequence information relating to a genome sequence;
   an inversion unit that generates, on the basis of the sequence information, inversion information in which the sequence is inverted; and
   a generation unit that generates, on the basis of the inversion information, protein information relating to a protein.
(2) The information processing apparatus according to (1), in which
   the sequence information is information relating to at least one of a sequence of amino acids, a sequence of DNA, or a sequence of RNA.
(3) The information processing apparatus according to (1) or (2), in which
   the generation unit includes
      a first prediction unit that predicts first protein information on the basis of the sequence information,
      a second prediction unit that predicts second protein information on the basis of the inversion information, and
      an integration unit that integrates the first protein information and the second protein information to generate the protein information.
(4) The information processing apparatus according to any one of (1) to (3), in which
   the protein information includes at least one of a structure of the protein or a function of the protein.
(5) The information processing apparatus according to (4), in which
   the protein information includes at least one of a contact map indicating a bond between amino acid residues forming the protein, a distance map indicating a distance between amino acid residues forming the protein, or a tertiary structure of the protein.
(6) The information processing apparatus according to (3), in which
   the integration unit executes machine learning using the first protein information and the second protein information as inputs to predict the protein information.
(7) The information processing apparatus according to (6), in which
   the first prediction unit executes machine learning using the sequence information as an input to predict the first protein information, and
   the second prediction unit executes machine learning using the inversion information as an input to predict the second protein information.
(8) The information processing apparatus according to (7), in which
   the integration unit includes a machine learning model for integration trained on the basis of an error between the protein information predicted using the first protein information for learning predicted using the sequence information for learning associated with correct answer data as a input and the second protein information for learning predicted using the inversion information generated on the basis of the sequence information for learning as an input as inputs and the correct answer data.
(9) The information processing apparatus according to (8), in which
   the first prediction unit includes a first machine learning model trained on the basis of an error between the first protein information for learning and the correct answer data, and
   the first machine learning model is re-trained on the basis of an error between the protein information predicted using the first protein information for learning and the second protein information for learning as inputs and the correct answer data.
(10) The information processing apparatus according to (8) or (9), in which
   the second prediction unit includes a second machine learning model trained on the basis of an error between the second protein information for learning and the correct answer data, and
   the second machine learning model is re-trained on the basis of an error between the protein information predicted using the first protein information for learning and the second protein information for learning as inputs and the correct answer data.
(11) The information processing apparatus according to (3), further including
   a feature amount calculation unit that calculates a feature amount on the basis of the sequence information, in which
   the generation unit generates the protein information on the basis of the feature amount.
(12) The information processing apparatus according to (11), in which
   the feature amount calculation unit calculates a first feature amount on the basis of the sequence information,
   the first prediction unit predicts the first protein information on the basis of the sequence information and the first feature amount, and
   the second prediction unit predicts the second protein information on the basis of the inversion information and the first feature amount.
(13) The information processing apparatus according to (11), in which
   the feature amount calculation unit calculates a first feature amount on the basis of the sequence information and calculates a second feature amount on the basis of the inversion information,
   the first prediction unit predicts the first protein information on the basis of the sequence information and the first feature amount, and
   the second prediction unit predicts the second protein information on the basis of the inversion information and the second feature amount.
(14) The information processing apparatus according to (12) or (13), in which
   the first prediction unit includes a first machine learning model trained on the basis of an error between the first protein information predicted using the sequence information for learning, which is associated with correct answer data, and the first feature amount for learning, which is calculated on the basis of the sequence information for learning, as inputs and the correct answer data.
(15) The information processing apparatus according to (12), in which
   the second prediction unit includes a second machine learning model trained on the basis of an error between the second protein information predicted using the inversion information generated on the basis of the sequence information for learning and the first feature amount for learning, which is calculated on the basis of the sequence information for learning, as inputs and the correct answer data.
(16) The information processing apparatus according to (13), in which
   the second prediction unit includes a second machine learning model trained on the basis of an error between the second protein information predicted using the inversion information, which is generated on the basis of the sequence information for learning, and the second feature amount for learning calculated on the basis of the inversion information as inputs and the correct answer data.
(17) The information processing apparatus according to any one of (11) to (16), in which
   the feature amount includes at least one of a secondary structure of the protein, annotation information relating to the protein, the degree of catalyst contact of the protein, or a mutual potential between amino acid residues forming the protein.
(18) The information processing apparatus according to any one of (1) to (17), in which
   the sequence information is information indicating a bonding order from an N-terminal side of amino acid residues forming the protein, and
   the inversion information is information indicating a bonding order from a C-terminal side of amino acid residues forming the protein.
(19) An information processing method executed by a computer system, including:
   acquiring sequence information relating to a genome sequence;
   generating, on the basis of the sequence information, inversion information in which the sequence is inverted; and
   predicting, on the basis of the inversion information, first protein information relating to a protein.
(20) A program that causes a computer system to execute the Steps of:
   acquiring sequence information relating to a genome sequence;
   generating, on the basis of the sequence information, inversion information in which the sequence is inverted; and
   predicting, on the basis of the inversion information, first protein information relating to a protein.
(21) The information processing apparatus according to any one of (11) to (17), in which
   the feature amount calculation unit calculates the feature amount by accessing a database in which the sequence information and the feature amount are associated with each other.

### REFERENCE SIGNS LIST

1 sequence information
2 protein information
4 information processing apparatus
5 acquisition unit
6 inversion unit
7 generation unit
10 inversion information
13 tertiary structure
14 contact map
15 distance map
18 first prediction unit
19 second prediction unit
20 integration unit
21 first contact map
22 second contact map
23 integrated contact map
26a machine learning model
26b machine learning model
26c machine learning model
29 sequence information for learning
34 inversion information for learning
35 first contact map for learning
36 second contact map for learning
42 feature amount calculation unit
43 sequence information feature amount
46 database
47 feature amount
50 sequence information feature amount for learning
53 inversion information feature amount
100 protein analysis system

1. An information processing apparatus, comprising:
an acquisition unit that acquires sequence information relating to a genome sequence;
an inversion unit that generates, on a basis of the sequence information, inversion information in which the sequence is inverted; and
a generation unit that generates, on a basis of the inversion information, protein information relating to a protein.

2. The information processing apparatus according to claim 1, wherein
the sequence information is information relating to at least one of a sequence of amino acids, a sequence of DNA, or a sequence of RNA.

3. The information processing apparatus according to claim 1, wherein
the generation unit includes
a first prediction unit that predicts first protein information on a basis of the sequence information,
a second prediction unit that predicts second protein information on a basis of the inversion information, and
an integration unit that integrates the first protein information and the second protein information to generate the protein information.

4. The information processing apparatus according to claim 1, wherein
the protein information includes at least one of a structure of the protein or a function of the protein.

5. The information processing apparatus according to claim 4, wherein
the protein information includes at least one of a contact map indicating a bond between amino acid residues forming the protein, a distance map indicating a distance between amino acid residues forming the protein, or a tertiary structure of the protein.

6. The information processing apparatus according to claim 3, wherein
the integration unit executes machine learning using the first protein information and the second protein information as inputs to predict the protein information.

7. The information processing apparatus according to claim 6, wherein
the first prediction unit executes machine learning using the sequence information as an input to predict the first protein information, and
the second prediction unit executes machine learning using the inversion information as an input to predict the second protein information.

8. The information processing apparatus according to claim 7, wherein
the integration unit includes a machine learning model for integration trained on a basis of an error between the protein information predicted using the first protein information for learning predicted using the sequence information for learning associated with correct answer data as a input and the second protein information for learning predicted using the inversion information generated on a basis of the sequence information for learning as an input as inputs and the correct answer data.

9. The information processing apparatus according to claim 8, wherein
the first prediction unit includes a first machine learning model trained on a basis of an error between the first protein information for learning and the correct answer data, and
the first machine learning model is re-trained on a basis of an error between the protein information predicted using the first protein information for learning and the second protein information for learning as inputs and the correct answer data.

10. The information processing apparatus according to claim 8, wherein
the second prediction unit includes a second machine learning model trained on a basis of an error between the second protein information for learning and the correct answer data, and
the second machine learning model is re-trained on a basis of an error between the protein information predicted using the first protein information for learning and the second protein information for learning as inputs and the correct answer data.

11. The information processing apparatus according to claim 3, further comprising
a feature amount calculation unit that calculates a feature amount on a basis of the sequence information, wherein
the generation unit generates the protein information on a basis of the feature amount.

12. The information processing apparatus according to claim 11, wherein
the feature amount calculation unit calculates a first feature amount on a basis of the sequence information,
the first prediction unit predicts the first protein information on a basis of the sequence information and the first feature amount, and
the second prediction unit predicts the second protein information on a basis of the inversion information and the first feature amount.

13. The information processing apparatus according to claim 11, wherein
the feature amount calculation unit calculates a first feature amount on a basis of the sequence information and calculates a second feature amount on a basis of the inversion information,
the first prediction unit predicts the first protein information on a basis of the sequence information and the first feature amount, and
the second prediction unit predicts the second protein information on a basis of the inversion information and the second feature amount.

14. The information processing apparatus according to claim 12, wherein
the first prediction unit includes a first machine learning model trained on a basis of an error between the first protein information predicted using the sequence information for learning, which is associated with correct answer data, and the first feature amount for learning, which is calculated on a basis of the sequence information for learning, as inputs and the correct answer data.

15. The information processing apparatus according to claim 12, wherein
the second prediction unit includes a second machine learning model trained on a basis of an error between the second protein information predicted using the inversion information generated on a basis of the sequence information for learning and the first feature amount for learning, which is calculated on a basis of the sequence information for learning, as inputs and the correct answer data.

16. The information processing apparatus according to claim 13, wherein
the second prediction unit includes a second machine learning model trained on a basis of an error between the second protein information predicted using the inversion information, which is generated on a basis of the sequence information for learning, and the second feature amount for learning calculated on a basis of the inversion information as inputs and the correct answer data.

17. The information processing apparatus according to claim 11, wherein
the feature amount includes at least one of a secondary structure of the protein, annotation information relating to the protein, the degree of catalyst contact of the protein, or a mutual potential between amino acid residues forming the protein.

18. The information processing apparatus according to claim 2, wherein
the sequence information is information indicating a bonding order from an N-terminal side of amino acid residues forming the protein, and
the inversion information is information indicating a bonding order from a C-terminal side of amino acid residues forming the protein.

19. An information processing method executed by a computer system, comprising:
acquiring sequence information relating to a genome sequence;
generating, on a basis of the sequence information, inversion information in which the sequence is inverted; and
predicting, on a basis of the inversion information, first protein information relating to a protein.

20. A program that causes a computer system to execute the Steps of:
acquiring sequence information relating to a genome sequence;
generating, on a basis of the sequence information, inversion information in which the sequence is inverted; and
predicting, on a basis of the inversion information, first protein information relating to a protein.
